(19) United States
(12) Patent Application Publication
(10) Pub. No.: US 2025/0268976 A1
(43) Pub. Date: Aug. 28, 2025

(54) BROAD-SPECTRUM PEPTOIDS FOR TREATMENT OR PREVENTION OF WOUND INFECTIONS, AND COMPOSITIONS AND METHODS OF USE THEREOF

(71) Applicant: Maxwell Biosciences, Inc., Austin, TX (US)

(72) Inventors: Sheetal Vali, Dallas, TX (US); Beth Burnside, Santa Rosa Beach, FL (US); Donald J. Treacy, Woodbine, MD (US); Edward M. Rudnic, Georgetown, TX (US); Joshua McClure, Austin, TX (US)

(21) Appl. No.: 19/057,783

(22) Filed: Feb. 19, 2025

Related U.S. Application Data
(60) Provisional application No. 63/557,217, filed on Feb. 23, 2024.

(51) Int. Cl. A61K 38/08; A61K 9/00; A61K 9/06; A61K 9/08; A61K 9/20; A61P 17/02; A61P 31/04; A61P 31/10; A61P 31/12

(52) U.S. Cl. CPC A61K 38/08; A61K 9/0019; A61K 9/06; A61K 9/08; A61K 9/2072; A61P 17/02; A61P 31/04; A61P 31/10; A61P 31/12

(57) ABSTRACT

Broad spectrum peptoids for treatment or prevention of wound infections, and compositions and methods of use thereof, are described.

### RELATED APPLICATIONS

This application claims the benefit of U.S. Provisional Application No. 63/557,217 filed Feb. 23, 2024, the contents of which is incorporated by reference herein in its entirety.

### TECHNICAL FIELD

The present disclosure relates to methods of preventing and treating infections in wounds using peptoid compounds.

### BACKGROUND

There is a need for improved treatment and prevention of infections in wounds, including but not limited to surgical site infections, burn wounds, and other types of wounds, such as treatment and prevention of infections of wounds in extreme filed conditions, such as on the battlefield, among others. For example, current treatment such as antibiotics are often of limited effectiveness in the battlefield due to treatment delays or ineffectiveness against pathogens, including gram positive and gram negative, high priority multidrug resistant pathogens. Preventing rapid formation of resistant biofilms requires immediate field treatment to prevent localized and systemic infections. The prevention of deep-seated biofilm infections should reduce the need for surgical intervention, morbidity, and mortality.

However, the development of new and improved anti-pathogenic compositions and methods of treatment and prevention of wound infections remains challenging.

### SUMMARY

The present disclosure relates in several embodiments to peptoid compounds, compositions and method of use thereof for treating or preventing wound infections.

According to a first aspect, the present disclosure relates in several embodiments to compositions for use in preventing or treating an infection in a wound, such as a wound in skin of a subject, the composition comprising one or more peptoid compounds in an amount of effective to prevent or treat the infection in the wound.

The compositions for use in preventing or treating an infection in a wound may include the following details, which can be combined with one another in any combinations unless clearly mutually exclusive:
(i) The one or more peptoids may be selected from: a peptoid compound H-Ntridec-NLys-Nspe-Nspe-NLys-NH₂; a peptoid compound H-Ntetradec-NLys-Nspe-Nspe-NLys-NH₂; and a combination thereof, wherein each peptoid is in an amount of effective to prevent or treat the infection in the wound.
(ii) The wound may be an acute wound, a chronic wound, a cut, an abrasion, a surgical incision, a skin graft, a traumatic wound, a hypertrophic scar, a diabetic ulcer, a venous ulcer, a pressure sore, a surgical wound, a post-operative wound, a burn wound, skin or tissue damage caused by radiation treatments, or any combination thereof.
(iii) The composition may be effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems, improve in the healing of skin and tissue damage caused by radiation treatments, reduce scar formation, or any combination thereof, following administration of the composition to a wound in skin of a subject.
(iv) The composition is effective to decrease wound closure time by up to 10%, 20%, 30%, 40%, 50%, 60%, 70%, 80%, 90%, 95%, 99%, or more.
(v) The composition may be effective to decrease wound healing time by up to 10%, 20%, 30%, 40%, 50%, 60%, 70%, 80%, 90%, 95%, 99%, or more.
(vi) The infection may an infection of a pathogen that has not been identified prior to the administration of the composition to the subject.
(vii) The infection may be a bacterial infection, a fungal infection, a viral infection, or any combinations thereof.
(viii) The bacterial infection may be an infection of a pathogen selected from Staphylococcus aureus, methicillin-resistant Staphylococcus aureus (MRSA) methicillin-susceptible Staphylococcus aureus (MSSA), coagulase-negative staphylococci, Klebsiella pneumoniae, Pseudomonas aeruginosa, Stenotrophomonas maltophilia, Citrobacter freundii, Streptococcus pneumoniae, Streptococcus pyogenes, Acinetobacter baumannii, Enterococcus faecium, Enterobacter cloacae, and any combinations thereof.
(ix) The fungal infection may be an infection of a pathogen selected from Candida krusei, Candida albicans, Paecilomyces variotti, Mucor racemosus, Rhizopus arrhizus, Rhizopus oryzae, Aspergillus fumigatus, Aspergillus niger, and any combinations thereof.
(x) The viral infection may be an infection of a pathogen selected from Coronaviruses (e.g., SARS-1, SARS-COV-2, Human beta Coronavirus), MERS, Influenza virus (e.g., Influenza H1N1, H3N2, Influenza H1N1, H3N2), Herpes Simplex Virus 1, Herpes Simplex Virus 2, Hepatitis B, Hepatitis C, Chikungunya (CHIKV), Dengue Fever virus, Ebola (EVD), HSV-1 acyclovir-resistant strains, Respiratory Syncytial Virus (RSV), Rift Valley Fever Virus (RVFV), Zika (ZIKV), and any combinations thereof.
(xi) The composition may be effective to prevent, decrease, or inhibit decrease a biofilm in the wound.
(xii) The composition may be formulated for topical administration, transdermal administration, transmucosal administration, intraperitoneal administration, subcutaneous administration, intramuscular administration, intravenous administration, or any combinations thereof, to the subject.
(xiii) The composition may be formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.
(xiv) The effective amount may be from 1-1000 mg/day, 25-750 mg/day, 50-500 mg/day, or 100-400 mg/day.
(xv) The composition may be formulated for administration one, two, three, or four times per day, once per week, once every two weeks, or once per month.

According to a second aspect, the present disclosure relates in several embodiments to a method of preventing or treating an infection in a wound in skin of a subject. The method comprises administering a composition of the present disclosure, in an amount of effective to prevent or treat the wound infection.

### BRIEF DESCRIPTION OF THE DRAWINGS

The present disclosure may be further understood through reference to the attached figures in combination with the detailed description that follows.

FIG. 1 is the molecular structure of peptoid compound MXB-24,656.

FIG. 2 is the molecular structure of peptoid compound MXB-22,510.

FIG. 3 is the molecular structure of peptoid compound MXB-27,369.

FIG. 4 is the molecular structure of peptoid compound MXB-25,605.

FIG. 5 is the molecular structure of peptoid compound MXB-24,816.

FIG. 6 is the molecular structure of peptoid compound MXB-25,739.

FIG. 7 is a Table summarizing example in vitro data of anti-bacterial properties of peptoids.

FIG. 8 is a Table summarizing example in vitro data of anti-fungal properties of peptoids.

FIG. 9 is a Table summarizing example in vitro data of activity against ESKAPE biofilms.

FIG. 10 is a graph reporting example relative luminescence at 6 hours post-infection of mice with bioluminescent Xen41 strain of P. aeruginosa in mice treated with peptoid compound MXB-22,510 or in untreated mice.

FIG. 11 is a graph reporting example quantification of number of Candida auris colony forming units per gram (CFU/g) in kidneys of mice intravenously challenged with Candida auris and treated with various doses of peptoid compound MXB-22,510 or in untreated mice.

FIG. 12 is a Table summarizing example data of peptoid activity against numerous viral pathogens.

FIG. 13 is a Table summarizing example data of Dengue virus 3 strain CO360/94 plaque-forming units/mol from a standard Vero cell assay, in cells treated with various peptoids.

FIG. 14 is a graph reporting example data of in vivo activity of peptoid compounds against SARS-COV-2 in the Syrian Hamster. Protection from weight loss was observed throughout the 7-day course of IN administration. Compounds: A=MXB-24,656; B=MXB-27,369.

### DETAILED DESCRIPTION

The present disclosure relates to peptoid compounds for preventing or treating infections in wounds. In some embodiments, the present disclosure relates to the use of peptoid compounds as broad-spectrum agents for treating or preventing wound infections and for promoting wound healing.

As described herein, in several embodiments, use of the peptoid compounds described herein for preventing or treating infections in wounds offers advantages including but not limited to having antibacterial, antiviral and antifungal properties in one molecule, and the additional benefit of preventing and disrupting biofilms, both bacterial and fungal. The peptoid compounds described herein also offer other advantages, such as inducing neutrophils to express anti-inflammatory cytokines, promoting apoptosis of damaged cells and supporting wound healing. An additional advantage is that one can treat a surgical site or wound without the delay of diagnosing specific pathogens. Therefore, the pathogen and strain agnostic peptoid compounds described herein can act in both a preventative and treatment wound setting.

In some non-limiting examples, there is a need for improved treatment and prevention of surgical site infections.

Infections related to surgical wounds, also referred to herein as Surgical site infection (SSI), are defined by the Centers for Disease Control and Prevention (CDC) as infection related to an operative procedure that occurs at or near the surgical incision within 30 days of the procedure, or within 90 days if prosthetic material is implanted at surgery is among the most common preventable complication after surgery. SSIs place sizable financial strains on healthcare systems worldwide, resulting in approximately 1 million additional inpatient days per year in the US alone. Infections can develop in surgical wounds due to various reasons, such as bacterial colonization, poor wound care, compromised immune response, and more. Surgical wounds and infections may include the following examples, without limitation.

Superficial Incisional Surgical Site Infection (SSI): This type of infection affects the outer layers of the incision site. It typically involves the skin and subcutaneous tissue and is usually caused by bacteria from the patient's own skin.

Deep Incisional Surgical Site Infection (SSI): This infection affects deeper tissue layers below the skin, such as muscle and fascia. It can be more serious and is associated with a higher risk of complications.

Organ/Space Surgical Site Infection (SSI): This infection occurs in any part of the body other than the incision site, which was opened or manipulated during surgery. Examples include infections within the abdominal cavity or the chest.

Cellulitis: A bacterial infection of the skin and underlying tissues, often resulting from contamination or improper wound care.

Abscess: A localized collection of pus within a tissue or organ, often requiring drainage.

Fasciitis/Necrotizing Soft Tissue Infection: A severe infection that spreads rapidly through the fascial planes and can lead to tissue necrosis.

Septicemia/Bacteremia: Systemic infection caused by bacteria entering the bloodstream from a surgical site or other source.

The most common pathogens isolated from infected surgical wounds are Staphylococcus aureus, coagulase-negative staphylococci, Streptococcus spp, and Enterococcus spp. Pathogenic organisms that cause infection also are known to form biofilms in or at the wound site. These biofilms act like shields, protecting the pathogens at the site of infection from both innate and exogenous circulating anti-microbial agents. Treatment of the wound therefore becomes challenging due to the presence of the biofilm shield, and presents a challenge for both innate and exogenous antimicrobial agents to reach the wound site, even at very high levels, in order to offer a treatment and cure the infection. Moreover, oftentimes a secondary pathogen infects such wounds, setting up a multi-pathogenic system that is incredibly challenging to treat.

The current standard of care for the prevention of infection is to administer preoperative antimicrobial agents when indicated based on published clinical practice guidelines and timed such that a bactericidal concentration of the agents is established in the serum and tissues when the incision is made. Empiric selection of antibiotics depends upon the initial gram stain, wound class, site of the wound, prior exposure to antibiotics, a history of colonization with antibiotic-resistant organisms (e.g., methicillin-resistant S. aureus [MRSA]), and local antimicrobial resistance patterns. Definitive antimicrobial treatment is guided by the clinical response of the patient and, when available, results of wound culture and sensitivities.

First-generation cephalosporin antibiotics (for example Cefazolin) are frequently used before surgical procedures. It covers a wide range of gram-positive bacteria, which are the most common culprits for surgical site infections. Cefuroxime is another cephalosporin antibiotic, often used in cases of penicillin allergies. It has a broader spectrum of activity against gram-negative bacteria compared to cefazolin. Clindamycin is used when there is a concern about anaerobic bacterial infections or when the patient is allergic to beta-lactam antibiotics. For bacterial resistant pathogens like methicillin-resistant Staphylococcus aureus (MRSA) infections, Vancomycin is effective against many gram-positive bacteria, including MRSA. Other antibiotics used in combination are Piperacillin-tazobactam and Ciprofloxacin-Levofloxacin. They cover a broad spectrum of bacteria, both gram-positive and gram-negative, as well as some anaerobes.

In addition to surgical site infections, in some non-limiting examples, there is a need for improved treatment and prevention of infection in other types of wounds.

Intravenous antibiotic administration is the current practice for wound infections. However, blood flow is often compromised in these wounds, which affects the ability for systemic antibiotics to reach the injury site in high enough concentrations. Furthermore, increasing the dose of antibiotics to adequately eradicate bacteria puts the patient at risk for toxicity to nontarget organs (e.g., kidney, liver, etc.). Local antibiotic therapies have provided a way to overcome poor perfusion issues in open fractures, but unfortunately infection still persists. It may be due to high concentrations of antibiotics not reaching the wound site quickly. Topically applied Vancomycin powder is gaining popularity as it has demonstrated efficacy in reducing surgical site infections when used prophylactically in spinal surgery, decreasing infection rates from 2.6% to 0.2% while dramatically reducing surgical costs. Despite this, there is little to no evidence for its use in a trauma scenario.

The misuse of traditional antibiotics like those named above, for example, can contribute to antibiotic resistance, so their appropriate use is crucial, thus in some cases necessitating the need for diagnostic testing to determine the right pathogen/treatment combination. The increasing prevalence of antibiotic-resistant bacteria poses a significant challenge in the treatment of SSIs and wound infections. Infections caused by drug-resistant pathogens can be harder to treat and may require more potent antibiotics leading to potential side effects. Deep-seated surgical site infections involving organs, deeper tissues or even bone, can be difficult to treat effectively due to limited penetration of antibiotics and challenges in accessing the infection site surgically.

In addition, another aspect of complexity to prevention and treatment of SSI's and wounds is the rapid formation of biofilms as bacteria that are within the wound multiply quickly and transform from the antibiotic-susceptible planktonic phenotype to the antibiotic-tolerant biofilm phenotype. This tolerance to antibiotics can occur within hours. Bacteria enter the wound in a planktonic, antibiotic-susceptible phenotype, attach to the surface of tissue, divide and grow in numbers until they reach a certain population density, and shift into an antibiotic-tolerant biofilm phenotype. These bacteria secrete extracellular polymeric substances that form a matrix of protein, polysaccharide, and extracellular DNA, which provides a barrier or shield, and protects them from being phagocytosed by host immune cells. Some of the cells, particularly the ones deep within the sessile community, reduce their metabolism and replication which makes them tolerant to antibiotic therapy. It has been shown that it can take as much as 1000-fold or greater levels of antibiotics to eradicate persister cells within a biofilm. The exact timing of biofilm formation and maturation depends on many factors such as the amount of initial colonization, environment, and bacteria species and strain. With that said, it has been shown that development of a biofilm occurs as early as 5 hours after inoculation, with maturation of this biofilm by 10 hours. After the systemic or local antibiotic therapy is finished, the persisting bacteria become metabolically active and replicate. This is believed to be one of the major reasons behind the high infection rates in open wounds.

Despite antibiotics being the standard of care regimen, infection rates remain high (between 11% and 28%). Providers continue to explore new advances in medicine to decrease infection in these complex wounds, and most importantly, target the prevention and disruption of biofilms.

Peptoids used at surgical site infections and wounds offer the advantage of antibacterial, and antifungal as one molecule, and the additional benefit of disrupting biofilms.

In another non-limiting example, there is a need for improved treatment and prevention of infections of wounds in extreme filed conditions, such as on the battlefield.

Antibiotics are often of limited effectiveness in the battlefield due to treatment delays or ineffectiveness against pathogens, including gram positive and gram negative high priority multidrug resistant pathogens. Preventing rapid formation of resistant biofilms requires immediate field treatment to prevent localized and systemic infections. The prevention of deep-seated biofilm infections should reduce the need for surgical intervention, morbidity, and mortality.

In some embodiments, the present disclosure relates to wound treatments advantageous for use in extreme field conditions to prevent wound infections and biofilm formation. In some embodiments, the peptoid compositions described herein can be used as pathogen-agnostic prophylactics that can prevent one or more of bacterial, fungal and viral planktonic and biofilm wound infections, thus combating the threat of severe, multidrug resistant infections requiring extreme medical procedures. In some embodiments, the present disclosure relates to use of peptoids as convenient pathogen-agnostic agents having a high margin of safety.

In some embodiments, the peptoids described herein can be used to treat or prevent wound infections while being coadministered with hemostatic, analgesic, and fluid replacement compounds through the various stages of battlefield wound care, as well as address delays in the care of penetrating injuries and prevention of deep-seated infections.

For example, as shown in the Examples of the present disclosure, in vitro and in vivo experimental results show activity of peptoid compounds against high priority pathogens, including but not limited to P. aeruginosa, K. pneumoniae, A. baumannii, S. aureus, as well as multiple additional pathogens, many of which are multidrug resistant (MDR). The peptoids described herein can conform to irregular wound shapes, can be self-administered, and can be removed with irrigation.

In another non-limiting example, there is a need for improved treatment and prevention of infections of burn wounds.

To date, burn wounds are not medically addressed in a uniform fashion. While cleansing, debridement, antimicrobial agent application, and dressings are all used, there is no consensus on which agent or dressing is optimal. One of the many reasons why antimicrobial agents are non-standard is because there are a variety of pathogens that can opportunistically infect a burn, including bacteria, fungi and viruses. Moreover, there is the nature and extent of the burn that determines the antimicrobial treatment and dressing type.

Burns and burn wounds are rapidly colonized, therefore closed dressing and infection control measures are a necessity. Dressing types are regional, customary and are often chosen based on cost. It would be advantageous to have a cost effective, broadly applicable antimicrobial agent, possibly embedded in a wound dressing that would universally be applied to all types of burns.

Accordingly, in some embodiments, use of the peptoid compounds described herein as part of the burn treatment regimen provides a pathogen-agnostic antimicrobial agent that is capable of preventing and treating a variety of infectious pathogens. The peptoids cover a broad range of enveloped organisms both gram positive and gram negative bacteria, as well as fungi and viruses. Because of the broad range of pathogen coverage, the need for diagnostic tests to determine the identity of the infectious agent may not be required.

Burns to the skin disrupt the body's ability to regulate water from escaping and from keeping infectious agents and dirt out. The skin is known as the integumentary system, and is the largest organ in the body. Damage to the skin as a result of buns requires specialized treatment.

There are several causes of burns: Thermal, chemical and radiation, all of which typically require different treatments.

There are also multiple of degrees of burns: First degree burn: superficial burn; epidermal layer of skin damaged, slightly pink skin, minor pain. Second degree burn: partial thickness burn; through the epidermis and into the dermis, blistering, red, warm to the touch painful. Third degree burn: full thickness burns; epidermis and all layers of dermis are burned. Characterized by cool touch due to lack of blood flow, charred or dry leathery skin, with nerve ending destroyed, with pain high in the surrounding area.

Burn treatment differs in the hospital setting vs austere environments (e.g., on the battlefield). In modern, hospital settings the burn is cleaned, treated and dressed according to protocol and degree of burn per facility. Infections are still a risk, even in the most sterile of environments, especially from MRSA and Pseudomonas aeruginosa.

Full thickness burns require excising the burned tissue within 24 hours to reduce the chances of infection, and requires grafting. In the hospital environment, Eschar (burned skin layer) is removed in the surgical suite to greatly reduce, or hopefully eliminate the chances of infection. However, in austere environments, burns are not able to be so ‘cleanly’ treated, and the eschar is generally left intact, leading to infection and possibly (likely) sepsis.

The key treatments for burns, while used with success, all have disadvantages. It is known that aggressive wound care is necessary, and that it includes topical agents (for moisture, etc.) used with antimicrobial agents, that has been associated with a reduced incidence of invasive wound infections. But the efficacy of topical therapy has not been definitively proven. The inflammatory response to burn complicates this infectious risk, as early signs and symptoms of sepsis are difficult to distinguish from basic burn response. Bacitracin, neomycin, or polymyxin B can be absorbed systemically, especially with third degree burns, and cause nephrotoxicity and neurotoxicity. Caution should be used in patients with impaired renal function and in those taking additional medications with nephrotoxic or neurotoxic adverse effects. An alternative is to limit the duration of use and/or to alternate with topical agents that are not absorbed. Silver sulfadiazine-Silver sulfadiazine cream (SSD 1%) (1) is used in many burn centers and is the standard of care in many burn centers for the treatment of burn wounds, For wounds covering more than 50 to 60 percent of the total body surface area, SSD does not consistently prevent or suppress bacterial growth, particularly of gram-negative bacteria. SSD is also known to interfere with re-epithelialization, so not advised in second or third degree burn situations. Nanocrystalline silver dressings are composed of a urethane film embedded with elemental silver that provides sustained release of silver into the wound. Compared with older silver formulations, it has stronger antimicrobial activity and longer-lasting properties that reduce dressing change frequency to weekly, depending upon the amount of exudate. However, some nanocrystalline silver dressings (e.g., Acticoat) require frequent moistening with water to maintain activation. In less developed countries or in austere environments both the cost and convenience of finding clean water present issues. Mafenide acetate is a potent carbonic anhydrase inhibitor, but has these adverse reactions associated with use of including metabolic acidosis, allergic reactions (e.g., rash, pruritus, hives, erythema, cosinophilia), and respiratory complications (e.g., tachypnea, decrease in arterial pCO2). Chlorhexidine gluconate is an antibacterial generally provided as a solution, or wash good for only superficial partial-thickness burns. While it does not interfere with healing it has limited utility with more severe burns. Honey is known as a good antimicrobial, and may aid with healing, but again, has disadvantages such as limited access in austere environments or for severe burn situations. Povidone iodine is a harsh antimicrobial that interferes with healing. Dakin's solution (0.025% sodium hypochlorite) is a broad-spectrum antimicrobial activity with efficacy in the clinical setting of MRSA, Vancomycin-resistant Enterococcus (VRE), and other antibiotic-resistant bacteria but has not been well studied for use in burns.

No single topical wound treatment is recognized as Standard of Care (SOC), and no topical wound treatment reviewed or addressed fungal or biofilm issues that are common pathogens that cause infections, and resistant infections, respectively.

In general, antibiotics are currently typically used to treat most wounds whether on the battlefield, in a surgical/hospital unit, or at home. Antibiotic resistance, or complete ineffectiveness against bacterial or fungal pathogens and biofilms, is increasing. The other major challenge today is that drugs have to be targeted to the specific pathogen increasing the time to treat if the diagnosis is wrong or incomplete.

In some embodiments, the present disclosure relates to the use of peptoid compounds described herein as pathogen-agnostic prophylactics that prevent bacterial, fungal and viral wound infections, as well as the ability to disrupt and prevent biofilm formation, thus combating the threat of resistant and severe infections requiring extreme medical procedures. In some embodiments, the present disclosure relates to the use of broad-spectrum peptoids with the ability to prevent wound infections due to ESKAPE pathogens.

In addition to the warfighter needs, in the civilian setting an increase in the elderly population, traumatic injuries, surgical procedures and a significant increase in obesity and diabetes have all led to higher rates of acute, chronic and surgical wounds.

Accordingly, in some embodiments, the present disclosure relates to a composition for use in preventing or treating an infection in a wound in skin of a subject, the composition comprising an amount of one or more peptoid compounds effective to prevent or treat the infection in the wound.

The peptoid compositions described herein may be optimized for easy use in extreme field conditions to eradicate SSI's and/or other wound infections. The peptoids can act as pathogen-agnostic treatments and/or prophylaxes to eradicate bacterial and fungal pathogens and have the ability to disrupt and prevent biofilm formation in wounds. The peptoids described herein can be formulated or co-formulated in a variety of dosage forms for use in SSI's and wound treatments.

The terms “peptoid” or “peptoid compound” as used herein refers to a type of biomimetic molecule that is similar to peptides but differs in its structure. Peptoids are synthetic oligomers composed of N-substituted glycine units. Accordingly, peptoids are also known as poly-N-substituted glycine compounds. In contrast to peptides, which have a peptide bond between amino acids, peptoids have a N-substituted (or N-alkylated) amide bond. This structural difference gives peptoids unique properties compared to peptides. Peptoids can be designed and synthesized to mimic the functions of natural peptides but with enhanced stability and different chemical properties. Peptoid compounds may be cyclic or linear. Peptoids have been described, for example, in U.S. Pat. Nos. 8,445,632, 8,828,413, 9,315,548, 9,872,495, 9,938,321, and International Patent Application Publication No.'s WO2021046562, WO2020223581, WO2021127294, WO2023287570, WO2022120393, and WO2021231343, the disclosures of which are incorporated herein in their entireties.

For example, without limitation, in some embodiments a peptoid compound may have a formula:

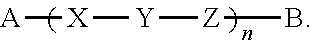

In such a compound, A can be selected from H and a terminal N-alkyl substituted glycine residue, where such an alkyl substituent can be selected from about C4 to about C20 linear, branched and cyclic alkyl moieties; n can be an integer selected from 1-3; B can be selected from NH₂, and one and two N-substituted glycine residues, such N-substituents as can be independently selected from α-amino acid side chain moieties and structural/functional analogs thereof; and X, Y and Z can also be independently selected from N-substituted glycine residues, such N-substituents as can be independently selected from α-amino acid side chain moieties and structural/functional analogs thereof and proline residues. Such X-Y-Z periodicity can provide such a compound a certain amphipathicity. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain-such homologs as would be understood in the art, including but not limited to plus or minus 1 or 2 or more methylene and/or methyl groups.

A can be H, and B can be selected from one or two N-substituted glycine residues, such a selection as can reduce the hydrophobicity of such a compound, as compared to compounds of 3-fold periodicity. In certain such embodiments, X can be an NLys residue; n can be 2-3; and B can be two N-substituted glycine residues. Without limitation, such a compound can be of a formula:

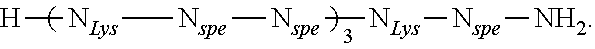

Regardless of identity of A, X and B, at least one of Y and Z can be a proline residue. X, Y and Z can be proline residues.

In certain other embodiments, A can be a terminal N-alkyl substituted glycine residue, with such an alkyl substituent as can be selected from about C6 to about C18 linear alkyl moieties. Regardless, B can be NH2, and n can be selected from 1 and 2. In certain such embodiments, A can be a terminal N-alkyl substituted glycine residue, with an alkyl substituent selected from about C6 to about C18 linear alkyl moieties. Regardless, B can be an NLys residue, and n can be 1.

In some embodiments, a peptoid compound may have a formula:

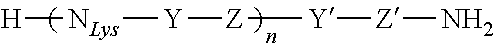

wherein n can be selected from 2 and 3; and Y, Z, Y′ and Z′ can be independently selected from N-substituted glycine residues, where such substituents can be independently selected from α-amino acid side chain moieties and carbon homologs thereof. Such Y′ and Z′ residues can be selected to provide such compound reduced hydrophobicity as compared to a compound of 3-fold periodicity. In certain such embodiments, at least one of X and Y can be a proline residue. Regardless, n can be selected from 2 and 3, and Y′ can be an N_{Lys} residue. In certain such embodiments, one or both X and Y can be proline residues. Without limitation, such a compound with reduced hydrophobicity can be of a formula:

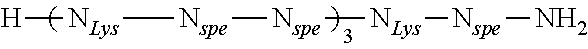

In some embodiments, a peptoid compound may have a formula:

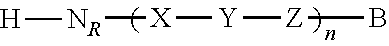

wherein B can be selected from NH₂ and X′; X, Y, Z and X′ can be independently selected from N-substituted glycine residues, where such substituents can be independently selected from α-amino acid side chain moieties and carbon homologs thereof; n can be an integer selected from 1 and 2; and R can be an N-alkyl substituent of such a glycine residue, as can be selected from about C₄ to about C₂₀ linear, branched and cyclic alkyl moieties. In some embodiments, n can be 2, and B can be NH₂. In some embodiments, n can be 1, and B can be X′. Accordingly, one or both of X and X′ can be N_{Lys} residues. Regardless, an alkyl substituent can be selected from about C₆ to about C₁₈ linear, branched and cyclic alkyl moieties, and X and X′ can be N_{Lys} residues. Without limitation, such a compound can be of a formula:

H—N_{tridec}-N_{Lys}—Nₛₚₑ—Nₛₚₑ-N_{Lys}—NH₂.

A peptoid may be a poly-N-substituted glycine compound comprising an N-terminus selected from H and an N-alkyl substituted glycine residue, where such an alkyl substituent can be selected from about C₄ to about C₂₀ linear, branched and cyclic alkyl moieties; a C-terminus selected from NH₂, one and two N-substituted glycine residues, such N-substituents as can be independently selected from α-amino acid side chain moieties and structural/functional analogs thereof; and 2 to about 15 monomeric residues between the N- and C-termini, each such residue as can be independently selected from proline residues and N-substituted glycine residues, said N-substituents independently selected from α-amino acid side chain moieties and structural/functional analogs thereof. Such monomers can be selected to provide such a compound a non-periodic sequence of monomers. As would be understood by those skilled in the art, such structural and/or functional analogy can be considered in the context of any such α-amino acid side chain, N-substituent and/or a sequence of such N-substituted glycine residues, such structure and/or function including but not limited to charge, chirality, hydrophobicity, amphipathicity, helical structure and facial organization. Such analogs include, without limitation, carbon homologs of such side chain-such homologs as would be understood by those skilled in the art, including but not limited to plus or minus 1 or 2 or more methylene and/or methyl groups.

The N-terminus of such a compound can be H; and the C-terminus can be selected from said one and two N-substituted glycine residues. A peptoid compound can comprise 2 to about 5 (X-Y-Z) non-periodic trimers. At least one of X, Y and Z in each of the trimers can be selected to interrupt 3-fold periodicity. Without limitation, at least one X in at least one said trimer can be an NLys residue. At least one of Y and Z in at least one such trimer can be a proline residue. The monomeric residues can comprise at least two non-consecutive of the same or repeat trimers, with at least one such residue therebetween to interrupt periodicity. At least one X in at least one such trimer can be an NLys residue, and at least one of Y and Z in at least one said trimer can be a proline residue.

The N-terminus of such a compound can be an N-alkyl substituted glycine residue, with an alkyl substituent selected from about C6 to about C18 linear alkyl moieties. A peptoid compound can comprise 2 to about 5 (X-Y-Z) non-periodic trimers. At least one of X, Y and Z in each of the trimers can be selected to interrupt 3-fold periodicity. The monomeric residues can comprise at least two non-consecutive of the same or repeat trimers, with at least one residue therebetween to interrupt periodicity. At least one X in at least one said trimer can be an NLys residue, and at least one of Y and Z in at least one said trimer can be a proline residue.

Various halogenated peptoids may be utilized in accordance with the teachings herein to make antiviral pharmaceutical compositions and treatments. These include, without limitation, various halogenated analogs of the foregoing peptoid compounds. These halogenated compositions may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. In some embodiments, the use of one or two bromo- or chloro-substitutions may be used. The peptoids described herein may be halogenated at various locations, for example and without limitation para halogenation on the peptoids containing aryl rings, ortho- and meta-substitution, or perhalogentation.

The peptoids described herein may be alkylated, for example and without limitation terminal alkylation. For example and without limitation, the alkyl substituent may be selected from about C₆ to about C₁₈ linear alkyl moieties.

In some embodiments, a peptoid may have antibacterial activity, antifungal activity, antiviral activity, or any combination thereof.

Without intending to be bound by theory, the peptoid compounds described herein mimic the structures and functions of antimicrobial peptides, key constituents of the human immune system, to exert broad direct antibacterial, antiviral and antifungal activity and wound healing properties. Peptoids are structural variants of peptides, in which the side chain groups are appended to nitrogen (instead of carbon) to form an amphiphilic molecule with both hydrophobic and cationic features. This novel structure resists proteolysis to form a more stable compound in vivo with the same anti-pathogenic properties as natural peptides.

Without intending to be bound by theory, antiviral activity of a peptoid may be associated with its ability to pass through a viral membrane and to bind to viral DNA or RNA. Furthermore, also without intending to be bound by theory, the mechanism of action may also feature disruption of membranes of various pathogens, by preferentially interacting with the lipid phosphatidylserine, which is found on the outer leaflet of various pathogen membranes. Phosphatidylserine is not typically present on mammalian cell surfaces, allowing peptoid compounds to exhibit selectivity towards microbial cell types. The peptoid compounds described herein offer substantial pharmacological advantages over monoclonal antibodies and biological therapeutics: smaller size, low risk of off target effects, low manufacturing cost, anti-inflammatory properties, no cold chain requirement, high stability in vivo, and multiple mechanisms of action.

Various peptoid compounds may be utilized in accordance with the teachings herein to make pharmaceutical compositions and treatments, including without limitation the peptoid compounds described in the various patents and patent application publications described herein, which are incorporated herein in their entireties.

The peptoids described herein may be synthesized and provided by any suitable method known in the art, such as, for example and not by way of limitation, the method described in Example 1 of the present disclosure, or by methods described in the patents and patent application publications disclosed herein.

Various counterions may be utilized in forming pharmaceutically acceptable salts of the peptoids disclosed herein. In some embodiments, pharmaceutically acceptable salts of the peptoids disclosed herein may include sodium or hydrochloride salts.

In some embodiments, the present disclosure extends to the preparation of prodrugs and derivatives of the peptoids of the invention. Prodrugs are derivatives which have cleavable groups and become by solvolysis or under physiological conditions the peptoid of the invention, which are pharmaceutically active. Such examples include, but are not limited to, choline ester derivatives and the like, N-alkylmorpholine esters and the like. In some embodiments, the peptoid compounds provided herein may be prepared e.g., in crystalline form and may be solvated or hydrated. Suitable solvates include pharmaceutically acceptable solvates, such as hydrates, and further include both stoichiometric solvates and non-stoichiometric solvates.

In some embodiments, the present disclosure relates to a composition for use in preventing or treating an infection in a wound in skin of a subject, the composition comprising one or more peptoids described herein. In some embodiments, the peptoid may be one or more of the peptoids described in Table 1 in the Examples of the present disclosure, including but not limited to peptoid compounds referred to herein as MXB-24,656, MXB-22,510, MXB-27,369, MXB-25,605, MXB-24,816, MXB-25,739.

It has been surprisingly found that not all peptoid compounds may be suitable for use as broad-spectrum, pathogen-agnostic, agents for use in treating or preventing infections in wounds.

In some embodiments, the peptoid compound for use as broad-spectrum, pathogen-agnostic, agent for use in treating or preventing infections in wounds is referred to herein as MXB-22,510, having a sequence H-Ntridec-NLys-Nspe-Nspe-NLys-NH₂, and having a molecular structure:

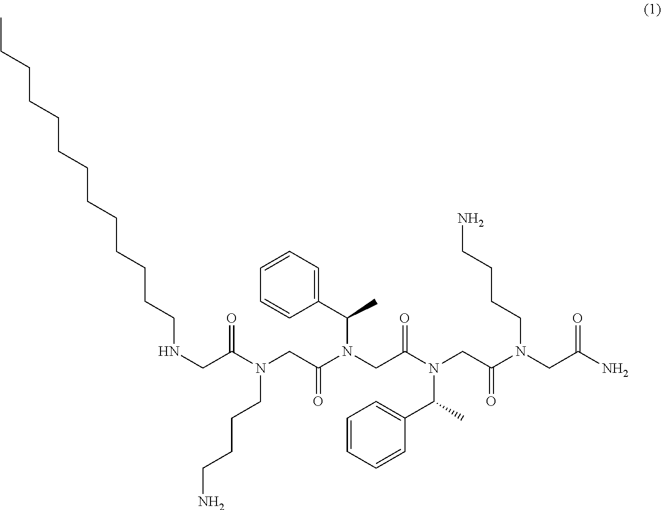 [SMILES: CCCCCCCCCCCCCNCC(=O)N(CCCCN)CC(=O)N(CC(=O)N(CC(=O)N(CCCCN)CC(N)=O)[C@H](C)c1ccccc1)[C@H](C)c1ccccc1]

The molecular formula of MXB-22,510 is C₄₇H₇₈N₈O₅ and the molecular weight of MXB-22,510 is 835.19 g/mol.

In some embodiments, the peptoid compound for use as broad-spectrum, pathogen-agnostic, agent for use in treating or preventing infections in wounds is referred to herein as MXB-24,816, having a sequence H-Ntetradec-NLys-Nspe-Nspe-NLys-NH₂, and having a molecular structure:

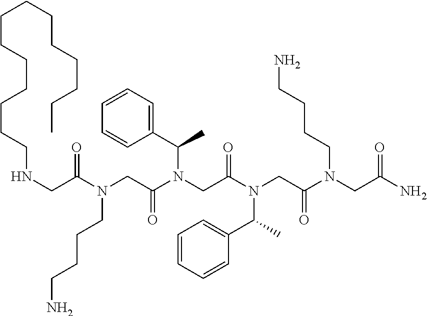 [SMILES: CCCCCCCCCCCCCCNCC(=O)N(CCCCN)CC(=O)N(CC(=O)N(CC(=O)N(CCCCN)CC(N)=O)[C@H](C)c1ccccc1)[C@H](C)c1ccccc1]

The molecular formula of MXB-24,816 is C₄₈H₈₀N₈O₅ and the molecular weight of MXB-24,816 is 849.22 g/mol.

As described in the Examples of the present disclosure, it has been unexpectedly found that MXB-22,510 and MXB-24,816 show surprisingly broad anti-pathogenic effects that are not observed in other peptoids tested. Accordingly, in several embodiments, the present disclosure relates to the use of the broad-spectrum anti-pathogenic peptoids MXB-22,510 and MXB-24,816 to treat or prevent a wide array of pathogen infections in wounds in a pathogen-agnostic manner. Compared with MXB-22,510 and MXB-24,816, other peptoids tested to date have shown significantly greater specificity of their anti-bacterial, anti-fungal, and/or anti-viral effects. Accordingly, in some embodiments, the present disclosure relates to a composition for use in preventing or treating an infection in a wound in skin of a subject, the composition comprising a peptoid compound H-Ntridec-NLys-Nspe-Nspe-NLys-NH₂, a peptoid compound H-Ntetradec-NLys-Nspe-Nspe-NLys-NH₂, or a combination thereof, in an amount of effective to prevent or treat the infection in the wound.

In some embodiments, MXB-22,510 and/or MXB-24,656 have exceptionally broad activity and remarkable safety, when tested in various animal models and toxicology studies. Especially relevant is that MXB-22,510 and/or MXB-24,656 has shown activity against a broad range of bacteria, including gram positive and gram negative strains, as well as a broad range of fungi and even some viruses. MXB-22,510 and/or MXB-24,656 has also shown that it can prevent and disrupt biofilms, a major cause of concern for wound treatment. MXB-22,510 and/or MXB-24,656 have no significant activity against commensal microbiome bacteria, as demonstrated against human gut microbiome. The Examples of the present disclosure show example experimental data reporting the broad-spectrum activity of MXB-22,510 and/or MXB-24,656 against various pathogens.

Advantageously, the peptoids described herein have wound healing properties, associated not only with their infection-fighting properties, but also including but not limited to the following. The peptoids are not immunogenic, and so do not interfere with healing. Peptoids promote apoptosis of damaged cells, and promote neutrophil engagement at the wound site. Peptoids are also non-irritating, and do not require continued moistening. Furthermore, peptoids can be administered via various routes, including but not limited to topically and systemically, without nephrotoxicity, allowing two-sided healing of wounds.

In some embodiments, the wound may be or may include, without limitation, an acute wound, a chronic wound, a cut, an abrasion, a surgical incision, a skin graft, a traumatic wound, a hypertrophic scar, an ulcer, such as a diabetic ulcer or a venous ulcer, a pressure sore, a surgical wound, a post-operative wound, a burn wound, skin or tissue damage caused by radiation treatments, or any combination thereof.

In some embodiments, a wound may be a result of, for example and without limitation, an acute injury, laceration, impact, bruise, burn, lesion, surgical incision, skin graft, or any combinations thereof. In some embodiments, a wound may be a result of a chronic condition or disease such as but not limited to a diabetic ulcer.

In some embodiments, the composition of the present disclosure may comprise an amount of the one or more peptoid compounds described herein effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems (such as HIV/AIDS or undergoing chemotherapy), improve in the healing of skin and tissue damage caused by radiation treatments, reduce scar formation, or any combination thereof, following administration of the composition to a wound in skin of a subject.

In some embodiments, the composition of the present disclosure may comprise an amount of a peptoid compound effective to decrease wound closure time by up to 10%, 20%, 30%, 40%, 50%, 60%, 70%, 80%, 90%, 95%, 99%, or more.

In some embodiments, the composition of the present disclosure may comprise an amount of a peptoid compound effective to decrease wound healing time by up to 10%, 20%, 30%, 40%, 50%, 60%, 70%, 80%, 90%, 95%, 99%, or more.

Because of the broad-spectrum, pathogen-agnostic, activity of the peptoids, in some embodiments, the infection may be an infection of an unidentified pathogen at the time of administering the composition. In some embodiments, the composition of the present disclosure is adapted for administering to a subject for preventing or treating an infection in a wound in the subject, prior to identifying, or confirming the identity of, the pathogen, or the presence of a pathogen, in the wound of the subject. In some embodiments, the infection may be an infection of one or more unidentified or unconfirmed bacteria, fungi, or viruses, or any combinations thereof.

In some embodiments, the infection may be a bacterial infection, a fungal infection, a viral infection, or any combinations thereof.

The one or more peptoids may be effective in simultaneously treating infections involving the one or more pathogens, including but not limited to one or more bacteria, fungi, viruses, or any combinations thereof.

In some embodiments, the one or more pathogens may show multi-drug resistant properties.

In some embodiments, the bacterial infection may be an infection of one or more types of bacteria. For example, and without limitation, the bacteria may be selected from Staphylococcus aureus, methicillin-resistant Staphylococcus aureus (MRSA) methicillin-susceptible Staphylococcus aureus (MSSA), coagulase-negative staphylococci, Klebsiella pneumoniae, Pseudomonas aeruginosa, Stenotrophomonas maltophilia, Citrobacter freundii, Streptococcus pneumoniae, Streptococcus pyogenes, Acinetobacter baumannii, Enterococcus faecium, Enterobacter cloacae, and any combinations thereof.

The Examples of the present disclosure describe example data on anti-bacterial activity of peptoids.

In some embodiments, the fungal infection may be an infection of one or more types of fungi. For example, and without limitation, the fungal infection may be an infection of a pathogen selected from Candida krusei, Candida albicans, Paecilomyces variotti, Mucor racemosus, Rhizopus arrhizus, Rhizopus oryzae, Aspergillus fumigatus, Aspergillus niger, and any combinations thereof.

The Examples of the present disclosure describe example data on anti-fungal activity of peptoids.

In some embodiments, the viral infection may be an infection of one or more types of viruses. For example, and without limitation, the viral infection may be an infection of a pathogen selected from Coronaviruses (e.g., SARS-1, SARS-COV-2, Human beta Coronavirus), MERS, Influenza virus (e.g., Influenza H1N1, H3N2, Influenza H1N1, H3N2), Herpes Simplex Virus 1, Herpes Simplex Virus 2, Hepatitis B, Hepatitis C, Chikungunya (CHIKV), Dengue Fever virus, Ebola (EVD), HSV-1 acyclovir-resistant strains, Respiratory Syncytial Virus (RSV), Rift Valley Fever Virus (RVFV), Zika (ZIKV), and any combinations thereof.

The Examples of the present disclosure describe example data on anti-viral activity of peptoids.

In some embodiments, the composition of the present disclosure may comprise an amount of the one or more peptoid compounds described herein effective to prevent, decrease, or inhibit decrease a biofilm in the wound.

In some embodiments, the composition may be formulated for administration via one or more routes to the subject, in order to treat the wound. In some embodiments, the composition may be formulated for administration including but not limited to, topical administration, transdermal administration, transmucosal administration, intraperitoneal administration, subcutaneous administration, intramuscular administration, or intravenous administration to the subject.

In some embodiments, the present disclosure relates to a composition comprising one or more peptoid compounds described herein and one or more pharmaceutically acceptable excipients.

In some embodiments, the wound healing composition may be formulated for topical administration to the skin.

In some embodiments, the composition may be formulated to be rubbed, poured, sprinkled, or sprayed on, introduced into, or otherwise applied to the skin of a subject.

For example, and not by way of limitation, in some embodiments, a topical formulation of the present disclosure may be, or may comprise, a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof. In some embodiments, the topical wound healing composition may be formulated for disposition into a bandage. In some embodiments, the topical wound healing formulation may be disposed in a bandage.

In some embodiments, the wound healing composition may be formulated for other or additional routes such as for administration to the subject's systemic circulation (e.g., the subject's bloodstream).

The compositions described herein may be administered to a subject via topical administration and/or one or more systemic routes, optionally in combination with topical application to the skin.

Accordingly, in some embodiments, the peptoid compounds of the present disclosure may be formulated in a composition suitable for administration to the subject via various routes to treat or prevent the wound infection. Such compositions can be prepared in a manner known in the pharmaceutical art. The peptoid compounds described herein can be formulated into pharmaceutically acceptable compositions and dosage forms for administration to a subject. In some embodiments, the present disclosure relates to a composition comprising an effective amount of a peptoid compound described herein for use in a method of treating a subject for a wound infection. In some embodiments, the present disclosure relates to the use of the peptoids described herein for the preparation of medicaments or as medicaments, that may be used for treating an infection of a wound.

The present disclosure provides pharmaceutical compositions comprising one or more peptoids and a pharmaceutically acceptable medium, such as an excipient, carrier, or the like. The peptoids described herein may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

Generally, the peptoid compounds described herein are administered in a therapeutically effective amount. “Therapeutically effective amount” means the amount of a compound that, when administered to a subject for treating a disease, is sufficient to effect such treatment for the disease. The therapeutically effective amount of the peptoid compound may be determined by a physician, in the light of the relevant circumstances, including the condition to be treated, the chosen route of administration, the peptoid compound administered, the age, weight, and response of the individual subject, the severity of the subject's symptoms, and the like.

In some embodiments, the effective amount may be from 1-1000 mg/day, 25-750 mg/day, 50-500 mg/day, or 100-400 mg/day.

Moreover, these compositions may be administered in a single dose, multi-dose or controlled release fashion.

The term “administering”, “administered” and grammatical variants refers to the physical introduction of an agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intraocular, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include oral, topical, epidermal or mucosal route of administration, for example, intranasally, vaginally, rectally, sublingually or topically. Administering can also be performed, for example, once, a plurality of times, and/or over one or more extended periods.

In some embodiments, the administration may be one, two, three, or four times per day. In some embodiments, the administration may be once per week, once every two weeks, or once per month.

Formulation for topical administration may include, for example, dry powder formulation with a polymer to potentially extend residence time and drug release rate, spray on foam, topical gel, and an aqueous solution. These formulations can be dressed with a bandage or hemostatic gauze to maintain the formulation in place on the wound and to provide a protective barrier for wound healing. A powder formulation containing drug and polymer may be provided in a sachet or stick pack, where it could be administered directly to the wound or suspended in an aqueous solution for irrigation and administration. A simple powder formulation may be dissolved in an aqueous solution for irrigation and administration. A spray on foam formulation or a gel formulation may be administered via a small aerosol container. These product types provide for variable dosing and are suited for unpredictable conditions, i.e. windy, wet, varied temperatures, or may be used in a field hospital or tertiary care facility.

The pharmaceutical compositions disclosed herein may be manufactured as tablets, liquids, gels, foams, ointments or powders. In some embodiments, these compositions may be applied as microparticles or nanoparticles.

In some embodiments, intranasal compositions may comprise any pharmaceutically acceptable excipient, such as those approved in nasal spray formulations and listed in the Food and Drug Administration's Inactive Ingredient Database, or justifiable based on the Food and Drug Administration's Guidance for Industry: Nasal Spray and inhalation Solution, Suspension, and Spray Drug Products-Chemistry, manufacturing, and Controls Documentation. As would be understood by skilled persons, typically, the excipients used in intranasal formulations should be safe and compatible with nasal mucosa. Some common excipients used in intranasal products include buffers to maintain the pH of the formulation within an acceptable range, preservatives to prevent microbial contamination, surfactants to enhance drug absorption and distribution, stabilizers to maintain the stability of the formulation over time, solubilizers to improve the solubility of poorly soluble drugs, viscosity modifiers to control the viscosity of the formulation for better administration, and tonicity agents to adjust the osmolarity of the formulation to be close to that of nasal mucosa.

Compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for subjects, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the peptoid compound is usually a minor component (e.g., from about 0.01% to about 50% by weight) with the remainder being various vehicles or carriers and processing aids helpful for forming the desired dosing form.

Liquid forms suitable for oral administration may include, without limitation, a suitable aqueous or nonaqueous vehicle with buffers, suspending and dispensing agents, colorants, flavors and the like.

Solid forms may include, without limitation any of the following ingredients, or compounds of a similar nature: a binder such as microcrystalline cellulose, gum tragacanth or gelatin; an excipient such as starch or lactose, a disintegrating agent such as alginic acid, Primogel, or cornstarch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. The peptoid compound in such compositions is typically a minor component, often being from about 0.05% to 10% by weight with the remainder being the injectable carrier and the like.

Transdermal compositions are typically formulated as a topical ointment or cream containing the peptoid compound, generally in an amount ranging from about 0.01 to about 20% by weight. When formulated as an ointment, the peptoid compound may be combined with either a paraffinic or a water-miscible ointment base. The peptoid compound may be formulated in a cream with, for example an oil-in-water cream base. Such transdermal formulations may include additional ingredients to enhance the dermal penetration of stability of the peptoid compounds or the formulation.

The peptoid compounds of the present disclosure can be administered by a transdermal device. Accordingly, transdermal administration can be accomplished using a patch either of the reservoir or porous membrane type, or of a solid matrix variety.

The peptoid compounds of the present disclosure can be administered subcutaneously, including, without limitation, the use of syringe and needle injection, autoinjectors, pen injectors, needle-free injectors, subcutaneous infusion, jet injectors, patch pumps, pump infusion sets, implantable devices, subcutaneous depots, subcutaneous sustained release formulations, or any combinations thereof. The most traditional and widely used method of subcutaneous administration involves using a syringe and needle. Autoinjectors are pre-filled devices that automatically inject a set dose of a pharmaceutical composition when pressed against the skin. Examples include, without limitation, EpiPen for epinephrine and various biologic medications. Similar to autoinjectors, pen injectors are pre-filled devices that allow patients to self-administer a specific dose of a pharmaceutical composition. They are user-friendly and may have features like dose adjustment. Needle-free injectors use high pressure to administer a pharmaceutical composition through the skin without using a needle. Subcutaneous infusion may involve using an infusion pump to deliver a continuous or intermittent flow of a pharmaceutical composition into the subcutaneous tissue. Jet injectors use a high-pressure stream of liquid to penetrate the skin and deliver a pharmaceutical composition into the subcutaneous tissue. Patch pumps may adhere to the skin and contain a reservoir of a pharmaceutical composition, and it is absorbed through the skin over a period of time. Pump infusion sets may include a cannula or needle that is placed under the skin for continuous pharmaceutical composition delivery. Implantable devices may be used to provide sustained release of a pharmaceutical composition subcutaneously. Implantable devices may be surgically implanted and can deliver a controlled dose of a pharmaceutical composition over an extended period.

Subcutaneous depot release refers to the administration of pharmaceutical compositions in a way that allows for sustained and controlled release of a pharmaceutical composition from a depot or reservoir located in the subcutaneous tissue. This method may be used to provide a prolonged therapeutic effect, reducing the frequency of dosing and improving patient compliance. In some embodiments, a subcutaneous depot release formulation may include, without limitation, a solution, a suspension, or biodegradable matrix, that is introduced (e.g., injected) into the subcutaneous tissue. The formulation then forms a depot, a localized reservoir of a pharmaceutical composition, beneath the skin. The subcutaneous depot formulation may release an active substance, e.g. a peptoid, gradually over an extended period.

A subcutaneous depot formulation may include, without limitation, biodegradable matrices, liposomal formulations, polymeric microspheres or nanoparticles, hydrogels, PLGA (poly(lactic-co-glycolic acid)) microparticles, implantable devices, or any combinations thereof. In biodegradable polymers or matrices, over time, the matrix breaks down, releasing a pharmaceutical composition in a controlled manner. Liposomes, which are lipid vesicles, can encapsulate a pharmaceutical composition and provide controlled release. Such liposomal formulations may be injected subcutaneously to create a depot of a pharmaceutical composition. Microspheres or nanoparticles made of biocompatible polymers can encapsulate a pharmaceutical composition and release it slowly over time. These particles can be suspended in a liquid formulation and injected into the subcutaneous tissue. Hydrogels are water-containing gels that can hold and release a pharmaceutical composition. Injectable hydrogels can form depots in the subcutaneous tissue. PLGA microparticles comprise PLGA, a biodegradable polymer commonly used to create microparticles for sustained drug release. PLGA microparticles can be injected subcutaneously to form a depot. Some subcutaneous depot release systems involve implantable devices, such as osmotic pumps or reservoirs. These devices are typically placed under the skin during a minor surgical procedure and provide controlled release of a pharmaceutical composition for an extended period.

Example formulations and methods of sustained release subcutaneous administration of the peptoids and pharmaceutical compositions thereof described herein include those described in the following references, the contents of all of which are incorporated herein in their entireties: Judy Senior, Michael L. Radomsky. (2000). Sustained-Release Injectable Products. Boca Raton: CRC Press; Thambi T, Li Y, Lee D S. Injectable hydrogels for sustained release of therapeutic agents. J Control Release. 2017 Dec. 10; 267:57-66. doi: 10.1016/j.jconrel.2017.08.006. Epub 2017 Aug. 4. PMID: 28827094; Chan Y P, Meyrucix R, Kravtzoff R, Nicolas F, Lundstrom K. Review on Medusa: a polymer-based sustained release technology for protein and peptide drugs. Expert Opin Drug Deliv. 2007 July; 4(4): 441-51. doi: 10.1517/17425247.4.4.441. PMID: 17683256; Lou H, Feng M, Hageman MJ. Advanced Formulations/Drug Delivery Systems for Subcutaneous Delivery of Protein-Based Biotherapeutics. J Pharm Sci. 2022 November; 111(11): 2968-2982. doi: 10.1016/j.xphs.2022.08.036. Epub 2022 Sep. 2. PMID: 36058255; Sequeira JAD, Santos A C, Serra J, Estevens C, Seiça R, Veiga F, Ribeiro AJ. Subcutaneous delivery of biotherapeutics: challenges at the injection site. Expert Opin Drug Deliv. 2019 February; 16(2): 143-151. doi: 10.1080/17425247.2019.1568408. Epub 2019 Jan. 24. PMID: 30632401; Badkar AV, Gandhi R B, Davis S P, LaBarre M J. Subcutaneous Delivery of High-Dose/Volume Biologics: Current Status and Prospect for Future Advancements. Drug Des Devel Ther. 2021 Jan. 13; 15:159-170. doi: 10.2147/DDDT.S287323. PMID: 33469268; PMCID: PMC7812053; Vaishya R, Khurana V, Patel S, Mitra AK. Long-term delivery of protein therapeutics. Expert Opin Drug Deliv. 2015 March; 12(3): 415-40. doi: 10.1517/17425247.2015.961420. Epub 2014 Sep. 24. PMID: 25251334; PMCID: PMC4605535; Remington's Pharmaceutical Sciences, 17th edition, 1985, Mack Publishing Company, Easton, Pa. The above-described components for intranasal, orally administrable, injectable subcutaneous, or topically administrable compositions are merely representative. Other materials as well as processing techniques and the like that are suitable for administering the peptoids and pharmaceutical compositions described herein are identifiable by skilled persons upon reading the present disclosure.

The peptoid compounds described herein can be administered in sustained release forms or from sustained release or controlled drug delivery systems, delivered via oral, intramuscular, subcutaneous, or transdermal route. A description of representative sustained release materials and description of delivery systems can be found in Remington's Pharmaceutical Sciences and Modern Pharmaceutics.

In some embodiments, the formulations described herein may include one or more chelation agents. In some embodiments, the chelation agent may be an efficacious anti-calculus agent including, but not limited to, one or more of zinc, hexametaphosphates, and diphosphonates. In some embodiments, the formulations described herein may include one or more chelation agents selected from aminopolycarboxylic acids, citric acid, edetate disodium anhydrous, edetate calcium disodium anhydrous citrate salts, sodium gluconate, transferrins, polymers, and any combinations thereof. In some embodiments, the aminopolycarboxylic acids may be selected from the group consisting of tetraxetan (DOTA), nitrilotriacetic acid (NTA), Ethylenediaminetetraacetic acid (EDTA or EDTA acid), ethylene glycol-bis(β-aminoethyl ether)-N,N,N′,N′-tetraacetic acid (EGTA or egtazic acid), 1,2-bis(o-aminophenoxy) ethane-N,N,N′,N′-tetraacetic acid (BAPTA), pentetic acid, diethylenetriaminepentaacetic acid (DTPA) nicotianamine, ethylenediamine-N,N′-bis (2 hydroxyphenylacetic acid) (EDDHA), Ethylenediamine-N,N′-disuccinic acid (EDDS), and any combinations thereof.

The following non-limiting formulation examples illustrate representative pharmaceutical compositions that may be prepared in accordance with the present disclosure.

Formulation 1—Tablets. A compound of the present disclosure may be admixed as a dry powder with a dry binder in an approximate 1:2 weight ratio. Additional diluent may be added as necessary, and a minor amount of magnesium stearate is added as a lubricant. The mixture is formed into 150-1500 mg tablets (50-500 mg of active compound per tablet) in a tablet press.

Formulation 2—Capsules. A peptoid compound described herein may be admixed as a dry powder with a starch diluent in an approximate 1:1 weight ratio. The mixture is filled into empty capsule shells (50-500 mg of peptoid compound per capsule).

Formulation 3—Liquid. A peptoid compound described herein (50-500 mg) may be admixed with sucrose (1.75 g) and xanthan gum (4 mg) and the resultant mixture may be blended, passed through a No. 10 mesh U.S. sieve, and then mixed in water. Sodium benzoate (10 mg), flavor, and color are diluted with water and added with stirring. Sufficient water may then be added to produce a total volume of 5 mL.

Formulation 4—Tablets. A peptoid compound described herein may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Formulation 5—Injection. A peptoid compound described herein may be dissolved or suspended in a buffered sterile saline injectable aqueous medium to a concentration of approximately 0.1-5 mg/mL.

Formulation 6—Topical. Stearyl alcohol (250 g) and a white petrolatum (250 g) may be melted at about 75° C. and then a mixture of a peptoid compound described herein (1-100 g g) methylparaben (0.25 g), propylparaben (0.15 g), sodium lauryl sulfate (10 g), and propylene glycol (120 g) dissolved in water (about 370 g) may be added and the resulting mixture is stirred until it congeals.

Formulation 7—Intranasal. To prepare 1 L of a 25 mM phosphate buffer, dissolve 0.6 g of potassium phosphate dibasic and 2.93 g of potassium phosphate monobasic in 800 mL of deionized (DI) water. Second, slowly add 2 g of both glycerin and 2 g sorbitol while mixing with an overhead mixer. Stir until the solution is clear and free from undissolved particulates. Next, slowly add 50 g of hypromellose while stirring with an overhead mixer until dissolved for a 5% solution. Then, add 1 g of EDTA as a preservative for the multidose solution, and finally add 100 mg of the peptoid for a 0.01% (% w/v) solution. The solution is diluted to 1 L with DI water, the pH adjusted with a 1N NaOH or 1N HCl solution to a pH of 5.0-7.0 and stirred until a clear solution is obtained.

The peptoid may be included in the formulation over a range of 0.005%-5%. Alternate buffer agents include histidine buffer for pH control in the physiological range, and may be utilized over a molarity range of 10 mM-100 mM. Alternate viscosity increasing agents include, but are not limited to, carbomers, polyvinylpyrrolidone (PVP), hydroxyethylcellulose (HEC), and poloxamers, and may be present in a range of 2-10%. Osmolality increasing agents can also include, but are not limited to, sorbitol, sodium citrate, or dextrose, and may be included at 1-5%. Taste masking agents can include, but are not limited to, sucrose and/or other sugars and may be present at 1-5%. Preservatives may be included in the range of 0.05%-2%, and can also include, but are not limited to, benzalkonium chloride and sodium benzoate.

Formulation 8-Subcutaneous Injection. To prepare 1 L of a 25 mM phosphate buffer, dissolve 0.6 g of potassium phosphate dibasic and 2.93 g of potassium phosphate monobasic in 800 mL of deionized (DI) water. The solution is diluted to 1 L with DI water, the pH adjusted with a 1N NaOH or 1N HCl solution to a target pH of 6.5 (range 6.0-7.0) and stirred until a clear solution is obtained. Slowly add 1 g of peptoid and stir until completely dissolved for a target concentration of 1 mg/mL (0.1% w/v). This solution can be sterilized by using a 0.22 μm filter and stored in a sterile container with closure until use.

Additional example peptoid formulations are described in Example 18.

The peptoid may be included in the formulation over a range of 0.005%-5%. Alternate buffer agents include histidine buffer for pH control in the physiological range, and may be utilized over a molarity range of 10 mM-100 mM. Preservatives may be included in the range of 0.05%-2%, and can also include, but are not limited to, benzalkonium chloride and sodium benzoate.

In some embodiments, the compositions described herein may be formulated as mixtures of one or more peptoids. For example, these mixtures may comprise peptoids in various molar ratios, such as 0.01:0.99 to 0.99:0.01, or any ratio in between. In some embodiments, the effective amount may be from 1-1000 mg/day, with a preferred embodiment of 25-750 mg/day, or a more preferred embodiment of 50-500 mg/day, or an even more preferred embodiment of 100-400 mg/day.

In some embodiments, a composition may comprise a peptoid compound described herein in mixtures or combinations with other agents, such as known antibiotic, antifungal, or antiviral compounds. In some embodiments, the peptoid compounds of the present disclosure may act synergistically with the known antiviral compounds, so that the resulting composition demonstrates improved effectiveness.

In some embodiments, the present disclosure relates to a method of preventing or treating an infection in a wound in skin of a subject. The method comprises administering a composition described herein, in an amount of effective to prevent or treat the infection in the wound.

In some embodiments, the subject may be a vertebrate animal. In some embodiments, the subject may be a mammal. In some embodiments, the subject may be a primate. In some embodiments, the subject may be a human. In some embodiments, the methods disclosed herein have veterinary applications and can be used to treat non-human animals, such as wild, domestic, or farm animals, including, but not limited to, cattle, sheep, goats, pigs, dogs, cats, and poultry.

“Treating” or “treatment” of a wound infection refers, in some embodiments, to aiding in healing of the wound, ameliorating the wound (e.g., arresting or reducing worsening of the wound or at least one of the symptoms related to the wound). In some embodiments “treating” or “treatment” refers to ameliorating at least one physical parameter, which may not be discernible by the subject. In some embodiments, “treating” or “treatment” refers to modulating the wound, either physically, (e.g., stabilization of a discernible symptom), physiologically, (e.g., stabilization of a physical parameter), or both.

“Preventing” or “prevention” as used herein refers to a reduction in risk of acquiring a wound infection (e.g., causing at least one of the clinical symptoms of the infection not to develop in a subject not yet exposed to or predisposed or susceptible to the infection, and not yet experiencing or displaying symptoms of the disease).

In some embodiments, the term “treating” or “treatment” of a wound encompasses preventing or inhibiting an infection in a wound. In some embodiments, an infection may be a viral infection, a bacterial infection, a fungal infection, or any combination thereof.

In some embodiments, a compositions and methods described herein may be combined with other compositions and methods, including known wound care active ingredients, compositions and methods, either in the same composition, or administered separately. In some embodiments, the compositions and methods of the present disclosure may act synergistically with the other active ingredients, compositions and methods, so that the resulting compositions and/or methods demonstrate improved effectiveness.

In some embodiments, the compositions and methods described herein may be useful for applications as wound care products for example as consumer wound care products, such as over-the counter products, or products prescribed by a healthcare professional. In some embodiments, the compositions and methods described herein may be useful for applications in extreme environments such as in the battlefield, for military use, or in emergency scenarios, such as in first aid kits, wound care for home use, for clinical use, for first responder uses, and the like.

In some embodiments, the compositions described herein are non-toxic to human cells, show improved tolerability, improved efficacy, or any combinations thereof, compared to previously existing compositions and products for treating or preventing wound infections.

### EXAMPLES

The present examples are provided for illustrative purposes only. They are not intended to and should not be interpreted to encompass the full breadth of the invention.

### Example 1. Preparation of Peptoid Compounds

The peptoid compounds listed in Table 1 were prepared using a sub-monomer protocol, on Rink Amide MBHA Resin. Example sub-monomer protocols are described in Zuckermann, R. N., Kerr, J. M., Kent, S. B. H., & Moos, W. H. (1992) J. Am. Chem. Soc., 114, 10646-10647 and in U.S. Pat. Nos. 8,445,632 and 6,887,845, the entireties of which are incorporated herein by reference. The starting reagents are bromoacetic acid and a small set of primary amines that are readily available commercially. The crude peptoid products were then cleaved from the resin and sidechain protective groups were removed in one step by acidolysis. The resulting residue was then resolubilized and lyophilized twice to produce peptoids as a dry powder. The peptoid products were then purified by HPLC to produce peptoids in powder form, with hydrochloride as the counter ion. Peptoid compounds were stored as dry powder at −20° C. and protected from light prior to preparation of stock solutions.

TABLE 1
Peptoid compounds tested against various pathogens

| Peptoid | | Molecular | | |
|---|---|---|---|---|
| compound | | weight without | Molecular | |
| name | Peptoid Sequence | HCl salt (g/mol) | Formula | Counter Ion |
| MXB-24,656 | H-NLys-NSpe(p-Br)- | 1233.78 | C₅₂H₆₇Br₄N₉O₆ | Hydrochloride |
| | NSpe(p-Br)-NLys-NSpe(p- | (1343.15 with HCl | | |
| | Br)₅-NSpe(p-Br)-NH₂ | salt) | | |
| MXB-22,510 | H-Ntridec-NLys-Nspe-Nspe- | 835.19 | C₄₇H₇₈N₈O₅ | Hydrochloride |
| | NLys-NH₂ | (944.56 with HCl | | |
| | | salt) | | |
| MXB-27,369 | H-Ndec-NLys-Nspe-Nspe(p- | 1273.31 | C₆₄H₉₂Br₂N₁₀O₇ | Hydrochloride |
| | Br)-NLys-Npse- Nspe(p-Br)- | (1382.68 with HCl | | |
| | NH₂ | salt) | | |
| MXB-25,605 | H-Ndodec-NLys-Nspe-Nspe- | 821.17 | C₄₆H₇₆N₈O₅ | Hydrochloride |
| | NLys-NH₂ | (930.53 with HCl | | |
| | | salt) | | |
| MXB-24,816 | H-Ntetradec-NLys-Nspe- | 849.22 | C₄₈H₈₀N₈O₅ | Hydrochloride |
| | Nspe-NLys-NH₂ | (958.58 with HCl | | |
| | | salt) | | |
| MXB-25,739 | H-Ndec-NLys-Nspe-Nspe(p- | 1401.49 | C₇₀H₁₀₄Br₂N₁₂O₈ | Hydrochloride |
| | Br)-NLys-Nspe-Nspe(p-Br)- | (1547.32 with HCl | | |
| | NLys-NH₂ | salt) | | |

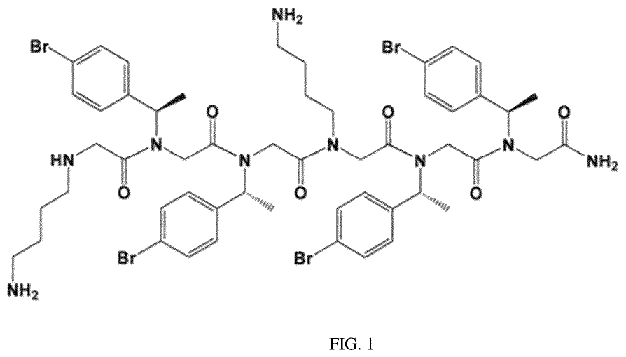

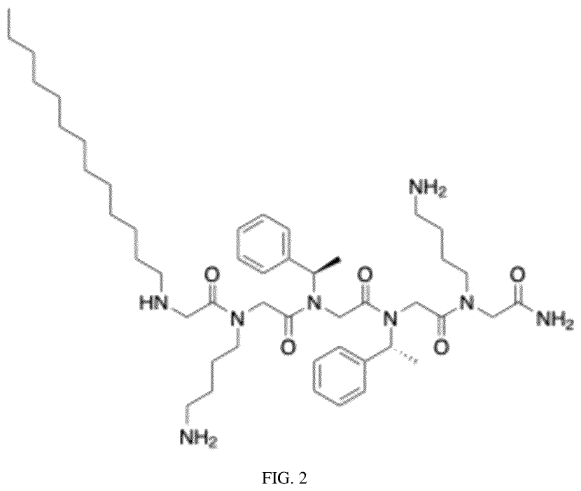

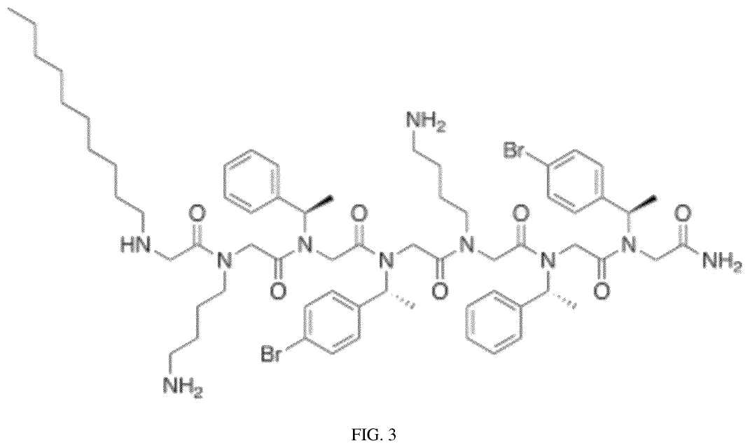

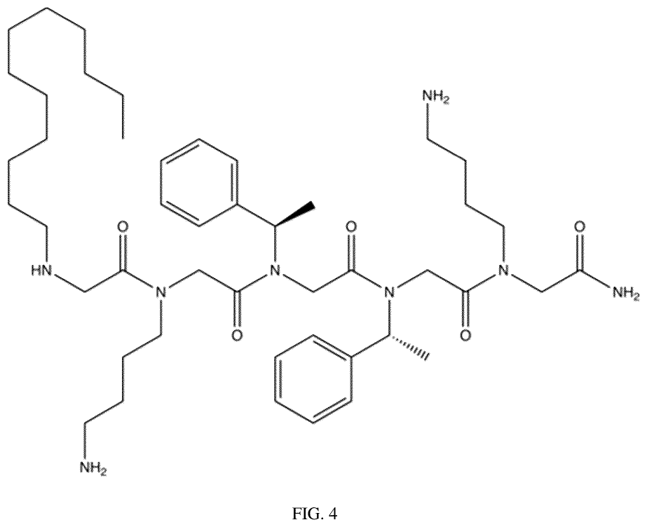

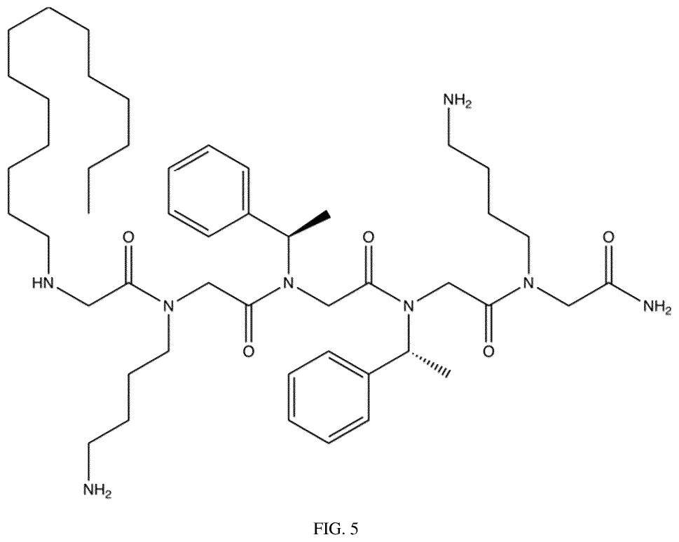

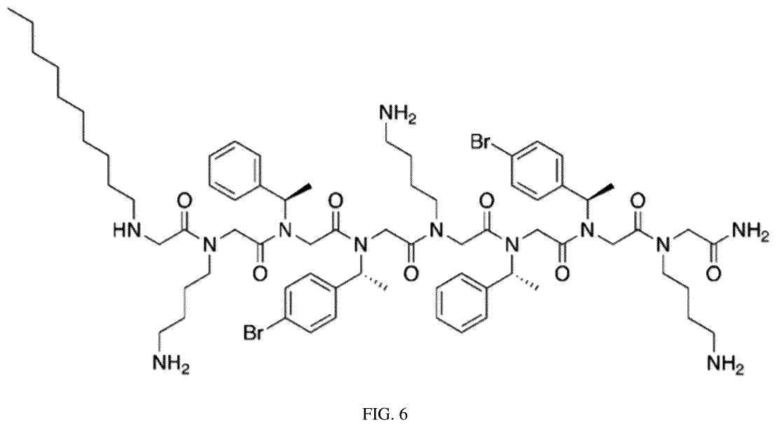

Chemical structures of the peptoid compounds listed in Table 1 are shown in FIG. 1-FIG. 6.

An initial stock concentration of each peptoid compound was prepared in tubes at 2 mg/ml in phosphate-buffered saline (PBS) pH 7.4 (Gibco; cat no. 10010023). Initial dissolution of lyophilized peptoid compound powders to create a stock solution was performed by gentle mixing by inverting the stock solution tube several times), followed by checking for turbidity, precipitation, or aggregate before proceeding to the next steps. If gentle inversion was insufficient to achieve a solution, the stock solution tube was briefly vortexed. The stock solution was then checked for any undissolved particulate, aggregates, or precipitation before proceeding to the next step. If gentle inversion and vortexing was insufficient to achieve a solution, the stock solution tube was briefly sonicated for 15-60 seconds. The stock solution was then checked again for any undissolved particulate, aggregates, or precipitation before proceeding to the next step. If turbidity, precipitation, or aggregate was observed at the initial stock concentration, the initial stock concentration was solubilized by diluting further in PBS to 1 mg/ml. Aliquots of the stock solutions were dispensed in polypropylene vials, protected from light, and stored at −20° C. or −80° C. prior to use.

Before testing, the aliquots were carefully observed for any signs of turbidity, precipitation or aggregate during sample preparation and were mixed, vortexed, or sonicated as needed.

### Example 2. Testing In Vitro Activity of Peptoid Compounds Against Bacterial and Fungal Pathogens

Peptoids in test groups A-F in Table 1 of Example 1 were tested in vitro for activity against several bacterial and fungal pathogens.

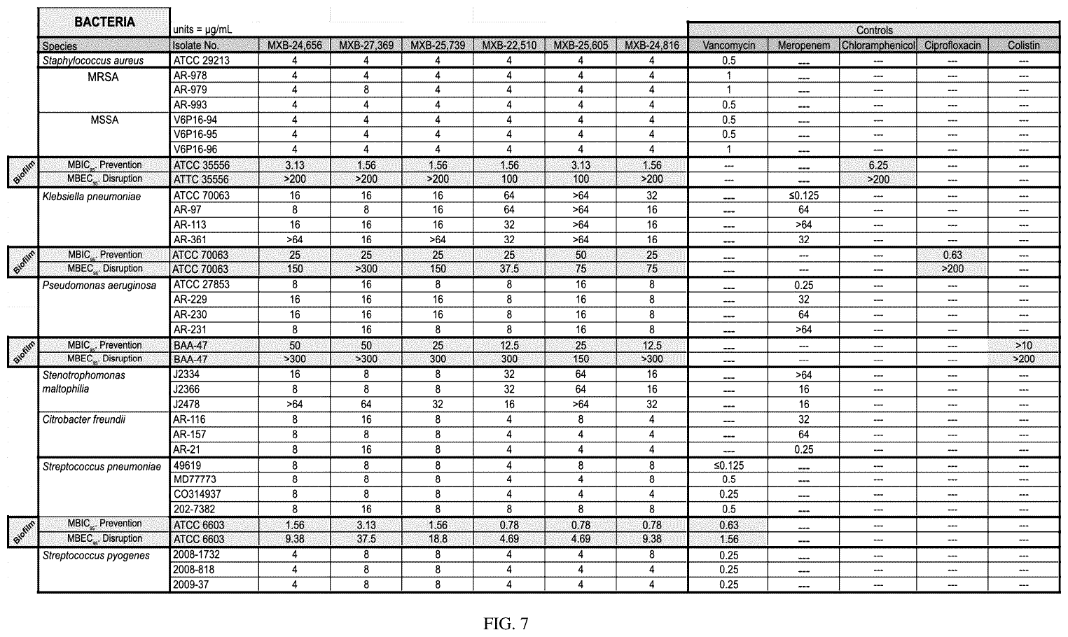

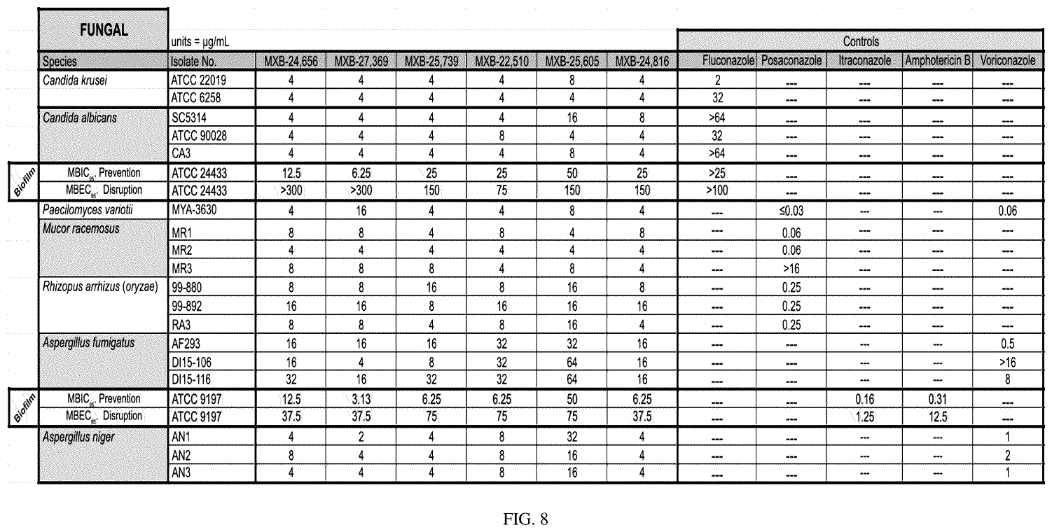

The results of anti-bacterial and anti-fungal activity for the tested peptoids is shown in FIG. 7 and FIG. 8, respectively.

In the Tables shown in FIG. 7 and FIG. 8, “MIC” refers to minimum inhibitory concentration, “MBIC” refers to minimum biofilm inhibitory concentration, and “MBEC” refers to minimum biofilm eradication concentration.

The peptoid compounds show in vitro activity against multiple bacterial and fungal pathogens for example but not limited to Staphylococcus aureus including both MRSA: methicillin-resistant Staphylococcus aureus, and MSSA: methicillin-susceptible Staphylococcus aureus and Aspergillus. As seen in FIG. 7 and FIG. 8, the peptoids tested achieved an MIC range of 4 μg/mL to 32 μg/mL depending on the species and strain tested. In addition, peptoids exert activity against biofilms, shown in FIG. 7 and FIG. 8.

Research continues to explore new advances in medicine to target the most complex wounds, most importantly target the prevention and disruption of biofilms. Peptoids show the inhibition of biofilm formation and disruption of established biofilms (FIG. 7 and FIG. 8). Peptoids exert direct bactericidal/fungicidal activity against the cell membrane, making retention of potency possible even when microbes are metabolically inactive, often associated with pathogen biofilms.

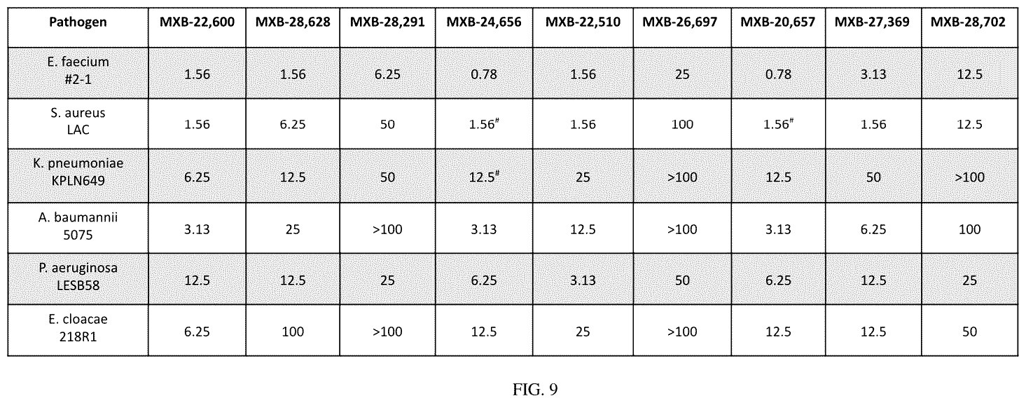

This also includes ESKAPE pathogens, (FIG. 9). The peptoids exert direct bactericidal/fungicidal activity against the cell membrane, making retention of potency possible even when microbes are metabolically inactive, often associated with pathogen biofilms.

### Example 3. In Vitro and In Vivo Antibacterial Efficacy vs Chemiluminescent Pseudomonas aeruginosa

MXB-22,510 shows in-vivo efficacy against Pseudomonas aeruginosa demonstrated utilizing the bioluminescent Xen41 strain of P. aeruginosa. Evaluation of the in vitro activity of peptoid compound MXB-22,510 using optical density, luminescence, and CFUs showed a minimal bactericidal concentration (MBC99) of 8 μg/mL.

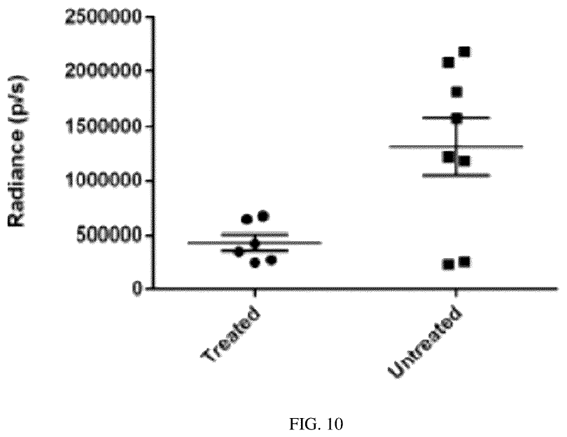

10x MIC was studied for in vivo activity against these bacteria following an intratracheally-introduced infection (10⁷ CFU) in mice. Mice were treated intratracheally with MXB-22,510 one hour post inoculation. Infected but untreated mice served as controls. At 6 hours post-infection both groups were evaluated for luminescence from the surviving bacteria (FIG. 10). Mice treated with MXB-22,510 showed significantly (p=0.0167) reduced average level of luminescence and 100% survival to study endpoint at 48 hours with no observable toxicity.

In a subsequent study, in vitro testing against clinical isolates of P. aeruginosa with various antibiotic susceptibility profiles, MXB-22,510 demonstrated consistent efficacy against all isolates with all strains tested showing MIC's of 8 μg/mL and MBC's of 16 μg/mL, and no cytotoxicity was observed up to 30-fold higher than the MIC. In vivo intratracheal administration also demonstrated significantly decreased bacterial CFU's in the lungs and spleen.

### Example 4. In Vivo Fungicidal Efficacy

In vivo efficacy of MXB-22,510 against Aspergillus niger conidia was also evaluated using C₅₇BL/6 mice that were immunosuppressed with Dexamethasone throughout the experiment. Mice were intranasally challenged with 8×10⁵ A. niger conidia on day 2 of immunosuppression and intratracheally treated with MXB-22,510 on day 3 and 4, and harvested on day 5. Samples were plated, incubated overnight at 37° C., and colony forming units (CFU) counted. Immunosuppressed mice challenged with A. niger conidia that were treated for 2 days had fewer significantly reduced CFU's in the lungs.

### Example 5. In Vivo Efficacy of MXB-22,510 Against Candida auris

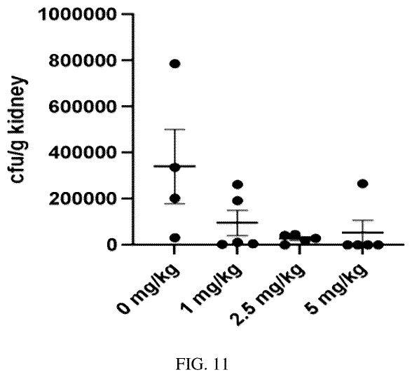

In vivo efficacy of MXB-22,510 against Candida auris was evaluated. Mice were intravenously challenged with 8.65E+07/mouse Candida auris and subcutaneously treated with MXB-22,510 (up to 5 mg/kg) after 3 hours of infection. At 24 hours post inoculation, the mice were sacrificed, samples harvested, and CFU's counted from kidney samples. MXB-22,510 had in vivo efficacy against Candida auris (FIG. 11). MXB-22,510 also demonstrated early data on synergy with standard of care antifungal agents.

### Example 6. In Vivo Efficacy of MXB-22,510 Against Herpes Simplex Virus-1 (HSV-1) in the Herpes Labialis Lip Scarification Mouse Model

In Vivo Efficacy of MXB-22,510 against Herpes Simplex Virus-1 (HSV-1) was studied in the herpes labialis lip scarification mouse model. Results: a decrease in lesion severity at the highest concentration, with almost no lesions visible by day 5 compared to buffer alone. In addition, prevention of HSV-1 from translocating to the trigeminal nerve, thus preventing latent viral infection. Viral infections are typically not the leading cause of resistant wound infections but often play the role of facilitator in wound infections.

### Prophetic Example 7. Evaluate Safety and Preliminary Efficacy Using Galleria mellonella (Waxworm) Infection Model

To perform preliminary safety testing in vivo peptoids will be analyzed for safety using the G. mellonella waxworm larvae model. G. mellonella are a widely used model for preliminary evaluation of antimicrobial agents due to their quick turnaround time to results and case of inoculation; they are also relatively inexpensive and not restricted by legal or ethical governing bodies. Furthermore G. mellonella serves as a valuable model for evaluating acute toxicity based on the Global Harmonizing System (GHS) of Classification and Labelling of Chemicals. The GHS ranks chemicals from lowest toxicity (category 5) to highest toxicity (category 1) and there is a strong correlation between GHS classification system and G. mellonella acute toxicity response. Taken in combination, these characteristics make G. mellonella a valuable tool to model novel antimicrobial safety and efficacy. In these toxicity studies G. mellonella will be selected based on weight between 200-300 mg and injected with one of the following treatment groups (1) 0.9% medical grade saline, (2) vehicle control, (3) lead peptoid candidate(s). The peptoids will be injected at MICs ranging from 256 μg/mL-0.25 μg/mL (10 mg/kg-0.01 mg/kg). Waxworm survival will be monitored and recorded for 3 days post-injection to determine live/dead counts.

Anticipated Results: In vivo safety and efficacy of peptoid compounds against an established waxworm model will be provided.

It is expected that MXB-22,510 and/or MXB-24,816 will show in vivo safety and efficacy in an established waxworm model.

### Prophetic Example 8. Ex Vivo Safety and Efficacy Studies—Testing Peptoid Efficacy and Toxicity in an Ex Vivo Human Skin Explant System

Peptoid cytotoxicity will be assessed using the ex vivo human skin model GenoSkin, a commercially available system that does not require IRB approval. The system uses real human skin (8-mm biopsy punch) that retains living cells for 7 days after surgery. The compounds will be applied to the system through 3 different routes: topical (compounds dropped directly on the skin surface), injection or systemic (added to the transwell that contains the tissue nutrient solution (FIG. 1B). The effect of the compounds on the skin tissues will be assessed by microscopy analyses: tissues will be fixed with freshly made 4% paraformaldehyde for 24 hours at 4° C., stored in 70% ethanol at room temperature, then paraffin embedded for sectioning and stained using a hematoxylin and eosin stain (H&E stain) for histology observations and compared to not treated tissues.

To assess efficacy, the GenoSkin tissue samples will be transferred into antibiotic-free media provided by the manufacturer (genoskin.com). Wounds will be infected with one of the SPAK strains and incubated in a humid environment at 37° C. with 5% CO2 in daily-fresh media. The compounds will be applied to the infected wounds 2 hours post-infection (HPI) and tissues collected between 1 and 7 days for CFU counts. Experiments will be conducted in 4 replicates for statistical analyses. Additionally, some of the tissues will be designated for histopathological evaluation and fixed in paraformaldehyde.

Anticipated Results: Data on tolerability and efficacy of peptoid compounds in a human skin ex-vivo model will be provided.

It is expected that MXB-22,510 and/or MXB-24,816 will show efficacy and tolerability in human skin ex-vivo model.

### Prophetic Example 9. Small Mammal In Vivo Efficacy Studies. Drug Product Formulation Development and Manufacture

Peptoids will be formulated for evaluation in murine and porcine models of infection. Multiple topical formulations will be developed with the intent of delivering drug to the wound site to minimize or prevent pathogens from colonizing the wound. Up to four (4) formulation approaches will be evaluated for ease of use and efficacy in the murine wound models. The lead formulation selected as a result of the murine model experiments, and a single formulation will be employed for the porcine model of infection.

Formulation approaches for topical administration will include dry powder formulation with a polymer to potentially extend residence time and drug release rate, spray on foam, topical gel, and an aqueous suspension. The approach for the dry powder polymer formulation will evaluate a dry and wet granulation process using standard pharmaceutical polymers such as hypromellose and polyvinylpyrrolidone as granulating and drug-release rate controlling agents. These formulations can be designed to release the anti-infective drug into a wound over the course of 24-48 hours. The approach for the spray on foam formulation will evaluate a solution/suspension containing poloxamer polymers that are in liquid state at room temperature and gel and 37° C. Experiments evaluating rate-controlling components in the foam formulation will also be conducted. The approach for the topical gel will be to start with a standard gel base formulation, and formulate to include the peptoid compound. The approach for the aqueous suspension will be evaluation of drug load/concentration, dispersing agents, surfactants, pH modifiers, and preservative agents. These formulations can be dressed with a bandage or hemostatic gauze to maintain the formulation in place on the wound and to provide a protective barrier for wound healing.

It is expected that drug product formulation prototypes (4) will be developed and manufactured using a peptoid (e.g. MXB-22,510 and/or MXB-24,816) for use in animal PK studies and in vivo animal safety and efficacy studies.

### Prophetic Example 10. Pharmacokinetic Characterization

Pharmacokinetic (PK) studies, which may include single as well as multiple drug dosing, are employed for determining the PK parameters in plasma and other organs as needed. The prototype formulations will be applied to a mouse wound model and the peptoid concentration will be measured as a function of time in both systemic circulation, as well as in local tissues. Plasma and tissue samples will be collected at several time points following drug administration. Samples will be prepared for liquid chromatography tandem mass spectrometry (LC-MS/MS) analysis and injected into the LC/MS/MS system for chromatographic separation. Drug concentrations will be extrapolated from previously prepared samples in which plasma and tissue from naïve mice are spiked with known amounts of test compounds. Study data will be gathered, and non-compartmental analysis will be conducted using the Phoenix/WinNonlin software. PK parameters such as T½, Cmax, Tmax, CL, MRT, V D, V ss, etc. as well as PK/PD parameters which quantify the activity of an antibiotic are the peak/MIC ratio, the T>MIC, and the 24 h-AUC/MIC ratio are assessed. In some cases and in support of drug design efforts, preliminary data for plasma drug concentrations may be assessed by using 10 μL of dried blood sample spots extracted from a tail nick of study mice.

Anticipated Results: Characterization of local and systemic pharmacokinetic performance from topical application of the peptoid compound in four different formulations in an animal model.

### Prophetic Example 11. Evaluation of Safety and Efficacy of Peptoids in a Murine Full Thickness Dorsal Wound Model Against Planktonic Bacteria and Bacterial Biofilms

Peptoids will be assessed in a full thickness dorsal wound model that has been established and validated against A. baumannii using 6-8 week old BALB/c mice and has subsequently been modified for other pathogens, including MRSA and P. aeruginosa (Rouse et al, Frontiers in Microbiology 2020, doi: 10.3389/fmicb.2020.00414). This model will allow testing of the peptoids for safety during infections using weights and clinical scores. Additionally, this model allows assessment of efficacy using two primary measures of wound healing: bacterial burden in the subject and time to wound closure.

On day-1 a SPAK pathogen will be grown overnight. On day 0 the bacteria are sub-cultured, grown to mid-exponential phase, and re-suspended in a 0.9% medical grade saline solution. Prior to infection the mice will be anesthetized via i.p. injection of 150 μl of ketamine/xylazine (13 mg/kg ketamine and 1 mg/kg xylazine) and given an IM injection of buprenorphine for pain management. The mouse dorsum will be shaved to remove all hair and then subsequently sterilized with chlorhexidine and 70% ethanol. A full thickness dorsal wound is made with a sterile 6 mm biopsy punch and inoculated with 50 μl of inoculum. The inoculum is allowed to dry for approximately 3 minutes prior to coverage with Tegaderm® to prevent contamination from entering the wound. One cohort will be wounded and have the wound sealed with Tegaderm® but uninfected as a wound healing control. From historical data (unpublished) at four hours post-infection pathogens will have established an infection in the wound. At this time the mice will be treated with (1) vehicle control, (2) a PFC-recommended antibiotic, (3) peptoid.

To assess the safety of the peptoid, weights and clinical scores will be taken every 24 hours for the first seven days and then every other day for the remainder of the experiment for all surviving cohorts. Mice from each cohort will be collected at 3, 6, and 21 days post-infection. The first two time points in this study (days 3 and 6) will be used to assess bacterial burden from the wound bed and spleen. Bacterial burden in the spleen is used to determine if bacterial dissemination away from the wound bed occurred. The final time point of the study, 21 days, will be used to assess wound healing, defined as the rate of the wound closure over time. Wound size will be measured every other day via an ARANZ™ instrument, which utilizes six lasers to measure wound area and depth. At predetermined timepoints for the cohorts where the experimental endpoint is defined to 21 days we utilize the bioluminescence produced by JE2::lux to quantify bacterial burden using an in vivo imaging system (IVIS Lumina XR, Caliper Life Sciences). Mice will be anesthetized via isoflurane gas and placed dorsal side up inside the IVIS® chamber. To determine the bacterial burden in each wound we will use a consistently-sized region of interest (ROI) to determine the total flux (photons/second) and report the change in flux throughout the course of the experiment.

Anticipated Results: Evaluation of safety and efficacy of the peptoid compound in four different formulations in a topical animal model of infection from planktonic bacteria.

It is expected that MXB-22,510 and/or MXB-24,816 will show in vivo safety and efficacy.

### Prophetic Example 12. Evaluation of Safety and Efficacy of Peptoids in a Murine Full Thickness Dorsal Wound Model Against Bacterial Biofilms

The murine full thickness wound model described above will be used for testing the antimicrobial efficacy of peptoids against pathogenic mature bacterial biofilms grown on polycarbonate discs. We will assess safety via weights and clinical scores, and we will assess efficacy by bacterial burden measurement (CFU counts). On Day 2 bacterial pathogens will be grown overnight in biofilm-forming conditions. On Day 1 the bacteria will be vortexed and homogenized into a single-cell suspension, subcultured, grown to mid-exponential phase, and used to inoculate a 4 mm diameter polycarbonate disc, previously made aseptic via UV sterilization. The bacterial impregnated polycarbonate disc is grown overnight on plates with biofilm media. Prior to surgery the mice will be anesthetized via i.p. injection of 150 μl of ketamine/xylazine (13 mg/kg ketamine and 1 mg/kg xylazine) and given an IM injection of buprenorphine for pain management. The mouse dorsum will be shaved to remove all hair and then subsequently sterilized with chlorhexidine and 70% ethanol. A full thickness dorsal wound will be made with a sterile 6 mm biopsy punch and inoculated with the impregnated 4 mm polycarbonate disc. The wound will be covered with Tegaderm® to prevent wound contamination. One cohort will be wounded and impregnated with a sterile polycarbonate disc and covered with Tegaderm® as a wound healing control. Four hours post-infection the mice will be treated with (1) vehicle control, (2) a PFC-recommended antibiotic, (3) peptoid. To further assess the safety of the product, weights and clinical scores will be taken every 24 hours for the first seven days and then every other day for the remainder of the experiment for all surviving cohorts. Mice from each cohort will be sacrificed at 3, 6, and 21 days post-infection, bacterial burden and wound healing will be measured as described above.

Anticipated Results: Evaluation of safety and efficacy of the peptoid compound in four different formulations in a topical animal model of infection from bacteria in a biofilm to help inform additional experiments.

It is expected that MXB-22,510 and/or MXB-24,816 will show in vivo safety and efficacy.

### Prophetic Example 13. Maximum Tolerated Dose and Neutropenic Thigh Infection Model Studies

Both acute and subacute non-GLP toxicity studies provide important information about the maximum tolerated dose (MTD) and the no observed adverse effect level (NOAEL). Each experimental group in the initial dose range-finding study consists of one male and one female mouse. To determine the MTD, up to ten dose levels per compound are tested in this experiment (vehicle and up to nine tested doses). These preliminary studies in 2 animals provide the necessary information about the acute toxic window, which is set to 1-3 days, while the observation period is no less than 15 days. Once MTD is assessed, it is verified in a confirmation group consisting of up to five male and five female mice (n=10) to ensure there is confidence in the dose-range study results. MTD verification studies serve as subacute toxicity studies, where animals are observed for up to 30 days. For both acute and subacute toxicity experiments, necropsy studies might be conducted, and organ specimens might be obtained for pathology studies in study animals immediately after they are euthanized.

Anticipated Results: Determination of the maximum tolerated dose of peptoid compound in topical application of 4 different formulation prototypes.

It is expected that MXB-22,510 and/or MXB-24,816 will show an efficacious MTD.

### Prophetic Example 14. Neutropenic Murine Thigh Infection Model

The neutropenic murine thigh infection, which is a standard model used by pharmaceutical companies and academic researchers, will be employed to assess the efficacy of peptoids against military-relevant, clinical isolates of ESKAPE (Enterobacter species, methicillin-resistant Staphylococcus aureus (MRSA), Klebsiella pneumoniae (KP), Acinetobacter baumanii (AB), Pseudomonas aeruginosa (PA), or Enterococcus species, as well as Escherichia Coli pathogens in mice. In short, two doses of cyclophosphamide are usually administered on days −4 and −1. Infection procedure, which consists in an intra-muscular injection of a 10⁵-10⁸ Colony-Forming Unit (CFU) inoculum contained in a 0.025 mL broth in the thigh of anesthetized mice, is completed 2 hours before drug administration. Dosing regimens consist of one or several drug doses given through different routes of administration throughout the usual 24 hr period following infection, but length of study can vary. Mice are euthanized at the study endpoint; CFU from each thigh are numerated for all study groups and a determination as to whether the bacterial load has diminished, increased, or remained stable over time is made.

Anticipated Results: Evaluation of safety and efficacy of peptoid compound in four different formulations in a well-established neutropenic animal model of infection to help inform additional experiments.

It is expected that MXB-22,510 and/or MXB-24,816 will show in vivo safety and efficacy.

### Prophetic Example 15. Large Mammal Efficacy Studies-Evaluate Efficacy of Peptoids in a Full Thickness Porcine Wound Model

Pigs will be used as a large mammal wound infection model to evaluate peptoid efficacy because pigs mimic human skin infections more accurately for several reasons. The first reason is the remarkable similarities between human and porcine skin anatomy. Both pigs and humans have skin that is firmly attached, a sparse hair coat, and thick dermal and epidermal layers whereas rodents lack all these features. Second both pigs and human wounds heal in the same manner via re-epithelization while mice use contractions around the injured area to stimulate wound healing. A full thickness formal wound porcine model using A. baumannii in Yorkshire pigs has been modified for MRSA and P. aeruginosa. On day-1 bacteria will be prepared as described above. A series of 16 wounds will be produced on the pig dorsum using a 10 mm diameter disposable biopsy punch to remove a ˜0.5 cm depth disk. Each is then covered by a transparent film cover (e.g., Tegaderm) to secure the wound. The dorsum is separated into 4 quadrants. There are 3-4 replicates for each treated wound to represent an experiment. On Day 0, each wound is infected with 50 μL of the pathogen and subsequently treated with peptoid. A 12 mm biopsy punch is made around each wound to homogenize tissues and enumerate CFUs. Blood will also be drawn to confirm the infection stays local and not septic.

Anticipated Results: Evaluation of safety and efficacy of peptoid compound in a formulation in a large mammal animal model of infection.

It is expected that MXB-22,510 and/or MXB-24,816 will show in vivo safety and efficacy.

### Example 16. In-Vitro and In-Vivo Antiviral Activity Data of Peptoids Versus Viral Pathogens

Peptoid antiviral activity has been observed against all enveloped viruses tested, including coronaviruses SARS-1, SARS-COV-2, Human beta Coronavirus (all three with mid-nanomolar EC₅₀ values), MERS, along with two strains of Influenza virus. In addition, peptoids have also shown activity against the enveloped viruses HSV 1 & 2 and Hepatitis B & C.

The list of enveloped viruses for which peptoids were studied and are active includes, without limitation: Chikungunya (CHIKV), Dengue Fever (Dengue virus 3 strain), Ebola (EVD), Herpes Simplex-1,2 (HSV), HSV-1 acyclovir-resistant strains, Hepatitis B, C (HBV, HCV), Influenza H1N1, H3N2, Respiratory Syncytial Virus (RSV), Rift Valley Fever Virus (RVFV), Zika (ZIKV).

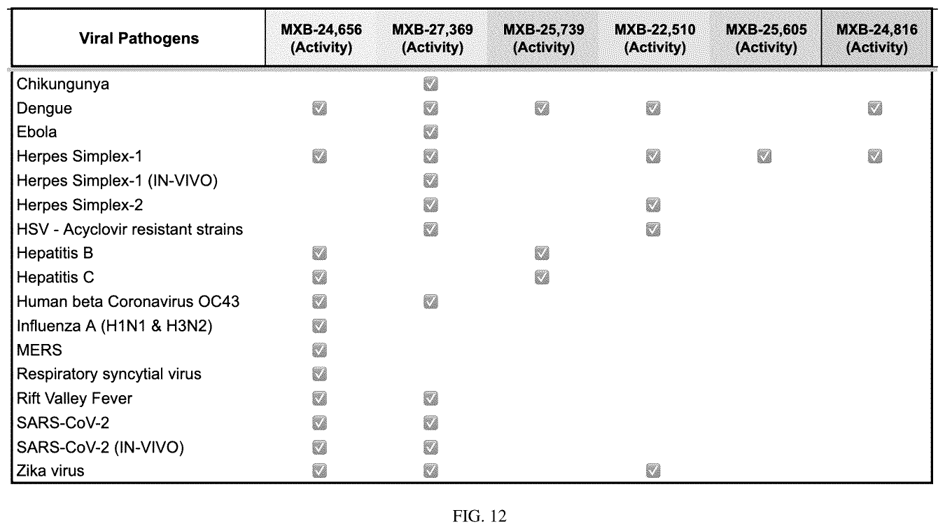

FIG. 12 reports example data of activity of peptoids tested against numerous viral pathogens, including resistant strains. Several have broad activity to key viral threats, and multiple compounds show activity to have broad anti-viral activity from various combinations of peptoids (Diamond, Gill; Molchanova, Natalia; Herlan, Claudine; Fortkort, John A.; Lin, Jennifer S.; Figgins, Erika; Bopp, Nathen; Ryan, Lisa K.; Chung, Donghoon; Adcock, Robert Scott; Sherman, Michael; Barron, Annelise E. Pharmaceuticals (2021), 14 (4), 304CODEN: PHARH2; ISSN: 1424-8247. (MDPI AG); Patrick M. Tate, Vincent Mastrodomenico, Christina Cunha, Joshua McClure, Annelise E. Barron, Gill Diamond, Bryan C. Mounce, and Kent Kirshenbaum ACS Infectious Diseases 2023 9 (8), 1508-1522).

Details of peptoids activity against various viral pathogens is discussed below.

### Dengue

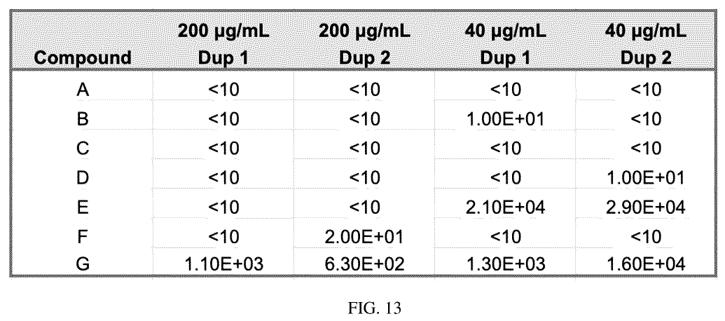

Peptoids showed virucidal activity of peptoids against Dengue virus 3 strain CO360/94. Following pre-incubation of the virus with peptoids at 40 μg/mL or 200 μg/mL, reduced virus concentration to below the limit of detection was observed for most of the compounds. Values shown in FIG. 13 represent plaque-forming units/ml from a standard Vero cell assay. DME control: without Maxwell compounds the concentration of infectious virus after 2 hours incubation was 3.25e4 per ml.

### Ebola (EVD)

Ebola virus disease (EVD) is caused by Ebola virus which belongs to the family Filoviridae virus. Peptoids were able to inhibit the ability of the Ebola virus to infect host cells. Following pre-incubation of the virus with peptoids at 100 μg/mL, no viral reproduction was observed over the 4-day time course of the study. Even at the lowest concentration evaluated, 20 μg/mL, viral titers could only be observed after 3 days.

### Herpes Simplex Virus-1 (HSV-1)

Peptoid compounds show activity against HSV-1 (Diamond, Gill; Molchanova, Natalia; Herlan, Claudine; Fortkort, John A.; Lin, Jennifer S.; Figgins, Erika; Bopp, Nathen; Ryan, Lisa K.; Chung, Donghoon; Adcock, Robert Scott; Sherman, Michael; Barron, Annelise E. Pharmaceuticals (2021), 14 (4), 304CODEN: PHARH2; ISSN: 1424-8247. (MDPI AG). HSV-1 infections cause recurrent oral lesions in the developed world. HSV-1 is also the primary cause of infectious blindness and genital infections in developed countries, and can lead to life-threatening infections in immunocompromised individuals. The in-vivo efficacy was evaluated in the herpes labialis lip scarification model. Results indicate a decrease in lesion severity at the highest concentration, with almost no lesions visible by day 5 compared with the control (buffer alone) mice. Reduction in viral load at this point in the infection was sufficient to allow the host defenses to completely remove the infection and heal the lesion. In addition, the inactivation prevented the virus from translocating to the trigeminal nerve, thus preventing latent viral infection.

Chikungunya. Rift Valley Fever & Zika

The antiviral activity of peptoids was studied against different RNA viruses (Chikungunya, Rift Valley Fever & Zika) (Patrick M. Tate, Vincent Mastrodomenico, Christina Cunha, Joshua McClure, Annelise E. Barron, Gill Diamond, Bryan C. Mounce, and Kent Kirshenbaum ACS Infectious Diseases 2023 9 (8), 1508-1522). Each virus was directly incubated with increasing concentrations of peptoids for 2 h. Virus-peptoid inoculum was collected and viral titers post-peptoid incubation were enumerated via plaque assay. Direct incubation of these viruses with the peptoid compounds reduced infectious virus titers to variable degrees. Most notably, all three enveloped viruses were susceptible to peptoid-mediated inactivation, whereas coxsackie B3 virus, a non-enveloped virus, was not affected by peptoid treatment.

### SARS-COV-2

In vitro studies with MXB-24,656 and MXB-27,369 demonstrated inhibitory activity of peptoids against several enveloped viruses, including SARS-COV-2. When incubated with virus at increasing concentrations, antiviral activity was observed, with approximate IC50 values of 20 μg/mL and 7 μg/mL, respectively.

In a NIAID sponsored study of peptoids in the SARS-COV-2 Syrian Hamster model the antiviral and disease-modifying activity of peptoids (MXB-24,656 and MXB-27,369) for the treatment of a SARS-COV-2 infection in wild-type hamsters were examined. Peptoids (0.1 and 1.0 mg/kg) were administered intranasally (IN) one hour prior to challenge with the virus.

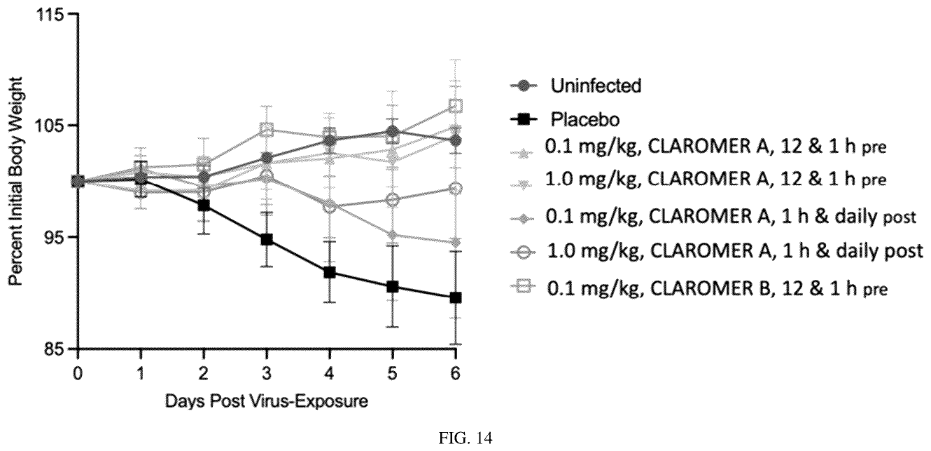

In-vivo protection and prophylaxis from SARS-COV-2 were observed within 24 hours from intranasal administration of a therapeutic dose. Absence of weight loss was observed for all animals treated prophylactically, indicating protection against infection from Day 1 throughout the 7-day course of the study (FIG. 14). Rapid activity is attained due to the direct virucidal mode of action of the peptoid compounds, indicating robust treatment against infection.

### Example 17. Example Peptoids Safety Data

A cytotoxicity study of peptoids was conducted using the EpiOral air-liquid interface system (a three-dimensional mucociliary tissue model consisting of primary cultures normal human-derived oral epithelial cells (EpiOral, MatTek Life Sciences, Ashland, MA, USA). Peptoids were incubated with the cells at 2-fold dilutions from 256 g/mL to 32 μg/mL for 3 hours at 37° C. At the end of the incubation, the supernatant was aspirated, cultures were rinsed, and 3-(4,5-dimethylthylthiazol-2-yl) (MTT) was added to assess cell viability. The extent of cell viability was assessed as the optical density of extracted samples measured at 570 nm. The results showed no observable cytotoxicity of peptoids as assessed by this metabolic assay, at concentrations up to 256 μg/mL.

A bacterial reverse mutation assay was conducted with peptoids using the plate incorporation method and bacterial strains Salmonella typhimurium TA98, TA100, TA1535, and TA1537 and Escherichia coli (E. coli) WP2uvrA in both the presence and absence of liver S9 obtained from rats induced with phenobarbital/β-naphthaflavone. Peptoids were evaluated for cytotoxicity at 0.005, 0.05, 5, 16, 50, 160, 500, 1600, and 5000 μg/plate and was found to produce >50% toxicity. Concentrations of peptoid showing acceptable bacterial survival produced no increase in revertant colonies exceeding that observed in DMSO controls by more than two-fold. Therefore, peptoids were considered to be non mutagenic under the conditions of this assay.

The ability of peptoids to cause the release of cytokines from human peripheral blood mononuclear cells (PBMCs) was evaluated. In this assay, PBMCs from a single donor were plated at a density of 2×10⁵ cells/well and incubated with Maxwell peptoids (20, 40, or 100 μg/mL) for 48 hours at 37° C. after which supernatants were collected and analyzed for the presence of a panel of cytokines (IFNγ, IL-1β, IL-2, IL-6, IL-8, IL-10, IL-17A, MIP-1α, TNFα). Anti-CD3 (15 μg/mL), superagonist anti-CD28 (15 μg/mL), lipopolysaccharide (100 ng/ml), and phytohemagglutinin (10 μg/mL) were used as positive controls. Peptoids did not cause the release of any of the pro-inflammatory cytokines evaluated.

Peptoids were evaluated for the potential for local adverse effects in nasal tissues using the MucilAir-Pool™ in vitro assay and repeated daily dosing for 4 days. No significant decrease in tissue integrity as measured by trans-epithelial electrical resistance (measured on Days 1-4) was observed. In addition, no decrease in mucociliary clearance frequency (measured on Day 4) or increases in cytotoxicity as measured by lactate dehydrogenase (measured on Days 2 and 4) were observed. A small decrease in ciliary beat frequency was observed on Days 2 and 4. Increases in inflammation-induced RANTES (Day 4) and GM-CSF (Day 2) release into the culture medium were also observed, but only at the highest concentration tested (100 μM). Further, no effects on several inflammatory cytokines (IL-8, IL-6, or β-defensin2; measured on Days 2 and 4) released into the culture medium were observed.

The toxicity and toxicokinetics associated with 7 days of repeated intranasal dosing of Sprague-Dawley rats with peptoids was evaluated. Rats were administered peptoid at a volume of 25 μL per nostril with both nostrils treated twice daily (i.e., total dose volume=50 μL/nostril or 100 μL/day) for total daily doses of 0 (vehicle; phosphate-buffered saline), 0.625, 2.5, and 10 mg/rat. All animals survived to their designated days of sacrifice. Potentially treatment-related clinical signs were limited to an increase in abnormal respiratory sounds. This sign was also noted in two vehicle control rats, as well as in five low dose (4M/1F), 8 middle dose (5M/3M), and 10 (5M/5F) high dose of the peptoid treated rats. There were no significant effects on rat body weights, but males at the high dose had body weights that were 11% less than the controls at necropsy. No treatment-related effects were observed at necropsy and no statistically significant effects on organ weights were detected.

### Example 18. Example Formulations of Peptoid Compounds

The peptoids described herein may be formulated into a wide variety of dosage forms for topical, targeted local delivery, or systemic delivery as may be required by the type of wound.

### Topical Formulation Approaches

Topical formulation approaches include powder, solution, suspension, semisolids, or infused into a bandage or other dressing material. Powder formulations include, but are not limited to, powder, granulation, pellets, or mini tablets. These powder dosage forms may be packaged or contained in a simple stick pack, sachet, vial, spray, shaker bottle, or multi-use bottle. The solution formulations may be provided as a solution, granules or powder for reconstitution, disintegrating tablet for dissolution and reconstitution, or incorporated into a spray bottle, with or without materials to provide a scaffold or topical bandage to protect the wound. The suspension formulations include, but are not limited to, aqueous suspension, suspension in another solvent, granules or powder for suspension, or disintegrating tablet for resuspension. These suspension dosage forms may be packaged or contained in a simple stick pack, sachet, vial, shaker bottle, or multi-use bottle. Semisolid formulation approaches include, but are not limited to, creme, gel, ointment, lotion, paste, balm, salve, emulsion, suppository (e.g. embedded in wax or polymer that liquifies at body temperature), spray, including spray on bandages, foam, including spray on foams, or film. Peptoids can also be infused into medical wound dressings including gauze, bandages, and wound packing, among others. Peptoids may be packed into a wound using a disintegrating tablet, drug eluting tablet or tablets, drug eluting beads or granules, or implantable, self dissolving sheet, wafer, block or suppository (e.g. embedded in wax or polymer that liquifies at body temperature), or thin wafer inserted for drug elution at the local wound site.

### Other Delivery Methods

In addition to topical formulation approaches, alternate administration approaches of the peptoids may be employed. These approaches include injection for local delivery, injection for systemic delivery, transdermal patch or other transdermal approach, intravenous, intranasal, intraocular, aural delivery, sublingual, buccal, or oral delivery, including immediate-release dosage forms, as well as modified release. Injection delivery methods may include intraperitoneal, subcutaneous, intramuscular, intrathecal, or intravenous.

### Example Powder Formulation

The peptoids may be administered as a simple powder as a standalone drug or with additional excipients to improve flowability or other processing requirements. This powder may be filled into a hard gelatin capsule and subsequently filled into bottles, or packaged in a sachet, stick pack, vial, or other container to aid in portability and ease of administration. This powder formulation may be applied directly to the wound, or dissolved in an aqueous vehicle for topical administration. A powder for topical administration may be prepared using the following formula:

### Formulation 1

Drug Substance—600 g (active ingredient)
Microcrystalline cellulose—100 g (processing aid-flowability)
Lactose—300 g (processing aid-flowability)

Step 1—blend the microcrystalline cellulose and lactose in a suitable blender and blend for 10 minutes

Step 2—add the drug substance and blend for an additional 10 minutes, or until the drug is uniformly distributed throughout the blender.

Step 3—discharge the powder blend from the blender into a suitable bin or container to store until the filling operation.

Step 4—fill the appropriate amount of blend into each package for storage, transfer, and administration.

### Example 2-Granulation

The drug substance powder can also be incorporated into a granulation that can produce a particle with improved flowability and density relative to the powder in Example 1. This granulation can be prepared either dry, or in the presence of water or other solvent. The binder may be added either wet (in the granulation solution) or dry with the rest of the materials. If water or other solvent is used, the blend is dried in a suitable pharmaceutical drier, such as a vacuum oven, forced air oven, or fluid bed drier. This granulation may be filled into a hard gelatin capsule and subsequently filled into bottles, or packaged in a sachet, stick pack, vial, or other container to aid in portability and ease of administration. This granulation formulation may be applied directly to the wound, or dissolved in an aqueous vehicle for topical administration. A granule formulation that may be used for topical application or dissolution into a topical solution may be prepared using the following formula:

### Formulation 2

Drug substance—700 g (active ingredient)
Microcrystalline cellulose—200 g (diluent and processing aid)
Povidone—100 g (binder)
Water (processing aid; removed during processing)

Step 1—add the active and excipients to a suitable pharmaceutical mixer or granulator such as a planetary mixer, high-shear granulator, fluid bed granulator, or extruder.

Step 2—slowly add the water while the mixer is operating until all the water has been added.

Step 3—continue the granulation step until the granulation endpoint is achieved.

Step 4—discharge the wet mass into a container suitable to hold the material until drying.

Step 5—charge the wet mass into a suitable drier and dry until the endpoint of less than 2% water is reached.

Step 6—discharge the dried granulation into a suitable bin or container to store until the filling operation.

Step 7—fill the appropriate amount of granulation into each package for storage, transfer, and administration.

### Additional Formulation Examples are Shown Below, and Follow a Similar Procedure for Preparation.

### Formulation 3

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 4

Drug substance—600 g (active ingredient)
Microcrystalline cellulose—300 g (diluent and processing aid)
Hydroxypropyl cellulose—100 g (binder)
Water (processing aid; removed during processing)

### Formulation 5

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

### Example 3—Pellets

The drug substance powder can also be incorporated into a pellet that can produce a particle with improved flowability and density relative to the powder in Example 1, and better flowability than the granulations in Example 2. These pellets may be filled into a hard gelatin capsule and subsequently filled into bottles, or packaged in a sachet, stick pack, vial, or other container to aid in portability and ease of administration. Pellet formulations that may be used for topical application or dissolution into a topical solution may be prepared using similar formulations to those shown in Example 2, with the addition of 2 processing steps. This pellet formulation may be applied directly to the wound, or dissolved in an aqueous vehicle for topical administration. Pellet formulation examples are shown here:

### Formulation 6

Drug substance—700 g (active ingredient)
Microcrystalline cellulose—200 g (diluent and processing aid)
Povidone—100 g (binder)
Water (processing aid; removed during processing)

### Formulation 7

Drug substance—700 g (active ingredient)
Microcrystalline cellulose—275 g (diluent and processing aid)
Povidone—75 g (binder)
Croscarmellose sodium—50 g (disintegrant)
Water (processing aid; removed during processing)

### Formulation 8

Drug substance—600 g (active ingredient)
Microcrystalline cellulose—300 g (diluent and processing aid)
Hydroxypropyl cellulose—100 g (binder)
Water (processing aid; removed during processing)

### Formulation 9

Drug substance—800 g (active ingredient)
Microcrystalline cellulose—125 g (diluent and processing aid)
Hydroxypropyl cellulose—50 g (binder)
Croscarmellose sodium—25 g (disintegrant)
Water (processing aid; removed during processing)

Step 1—add the active and excipients to a suitable pharmaceutical mixer or granulator such as a planetary mixer, high-shear granulator, fluid bed granulator, or extruder.

Step 2—slowly add the water while the mixer is operating until all the water has been added.

Step 3—continue the granulation step until the granulation endpoint is achieved.

Step 4—Load the wet mass into a suitable extruder and extrude using a screen with apertures between 300 μm and 800 μm.

Step 5—extrude the wet mass and introduce the extrudate into the marumerizer for pellet formation and spheronization.

Step 6—discharge the wet mass of pellets into a container suitable to hold the material until drying.

Step 7—charge the wet mass into a suitable drier and dry until the endpoint of less than 2% water is reached.

Step 8—discharge the dried pellets into a suitable bin or container to store until the filling operation.

Step 9—fill the appropriate amount of pellets into each package for storage, transfer, and administration.

### Example 4-Mini Tablets

The drug substance powder can also be incorporated into minitablets that can produce a particle with similar performance characteristics as a pellet. Minitablets offer another dry formulation approach, where a solvent may not be required. These minitablets typically have a diameter on the order of 500-2000 μm may be filled into a hard gelatin capsule and subsequently filled into bottles, or packaged in a sachet, stick pack, vial, or other container to aid in portability and ease of administration. This minitablet formulations may be applied directly to the wound, or dissolved in an aqueous vehicle for topical administration. Minitablet formulation examples are shown here:

### Formulation 10

Drug substance—600 g (active ingredient)
Microcrystalline cellulose—300 g (diluent)
Lactose—100 g (diluent)
Hypromellose (HPMC)—50 g (binder)
Colloidal silicon dioxide—50 g (glidant)
Magnesium stearate—5 g (lubricant)

### Formulation 11

Drug substance—600 g (active ingredient)
Silicified microcrystalline cellulose—400 g (diluent)
Hypromellose (HPMC)—50 g (binder)
Colloidal silicon dioxide—50 g (glidant)
Magnesium stearate—5 g (lubricant)

### Formulation 12

Drug substance—600 g (active ingredient)
Silicified microcrystalline cellulose—400 g (diluent)
Polyvinylpyrrolidone (PVP)—50 g (binder)
Colloidal silicon dioxide—50 g (glidant)
Magnesium stearate—5 g (lubricant)

### Formulation 13

Drug substance—700 g (active ingredient)
Silicified microcrystalline cellulose—250 g (diluent)
Polyethylene glycol (PEG)—50 g (binder)
Colloidal silicon dioxide—50 g (glidant)
Magnesium stearate—5 g (lubricant)

### Example 7—Cream

A topical cream formulation may be prepared using the following formula for the preparation of a batch of approximately 1 Kg:

### Formulation 14

Methyl paraben—0.25 g (preservative)
Propyl paraben—0.15 g (preservative)
Polysorbate 60—10 g (emulsifier)
Propylene glycol—120 g (viscosity modifier)
Stearyl alcohol—200 g (oleaginous phase)
White petrolatum—200 g (oleaginous phase)
Purified water—470 g (aqueous base)

Step 1—mix the stearyl alcohol and white petrolatum and heat to approximately 75° C. to melt the base.

Step 2—Dissolve the remaining excipients in the purified water by stirring until a solution is obtained.

Step 3—Add approximately 5 g of drug substance to the purified water solution and mix for 5 additional minutes to dissolve the drug to manufacture a 5% ointment.

Step 4—Slowly incorporate the aqueous solution to the oleaginous base and mix until it is well mixed.

Step 5—Fill the cream into a suitable package such as a tube or pump bottle.

### Example 8—Gel

Topical gels can be used for sustained-release of actives, provide lubrication, and a carrier of pharmaceutical agents. Hydrogels are water-based and are less oily than creams or ointments, as well as exhibit excellent spreading properties, and may exhibit a higher retention time on the skin. Hydrogels can be simple formulations and may provide for a higher drug capacity than oil based formulations due to the high aqueous solubility of the drug substance. Gel formulation examples are shown here:

### Formulation 15

Drug Substance—50 g (active)
Carbopol—300 g (polymer/viscosity)
Purified Water—650 g (solvent)

### Formulation 16

Drug Substance—50 g (active)
Sodium carboxymethylcellulose—400 g (polymer/viscosity)
Purified Water—550 g (solvent)

### Formulation 17

Drug Substance—50 g (active)
Hypromellose—200 g (polymer/viscosity)
Purified Water—750 g (solvent)

Step 1—Slowly add the polymer to the purified water while stirring slowly using a suitable mixer such as a Silverson mixer. Continue to mix until the polymer exhibits a lump-free dispersion.

Step 2—Slowly add the drug substance to the polymer dispersion and mix until dissolved.
Step 3—Fill the gel into a suitable package such as a tube or pump bottle.

### Example 9—Ointment

Hydrophilic ointment may be prepared using the following formula for the preparation of about 1 Kg of base:

### Formulation 18

Methyl paraben—0.25 g (preservative)
Propyl paraben—0.15 g (preservative)
Sodium lauryl sulfate—10 g (emulsifier)
Propylene glycol—120 g (viscosity modifier)
Stearyl alcohol—250 g (oleaginous phase)
White petrolatum—250 g (oleaginous phase)
Purified water—370 g (aqueous base)

Step 1—mix the stearyl alcohol and white petrolatum and heat to approximately 75° C. to melt the base.

Step 2—Dissolve the remaining excipients in the purified water by stirring until a solution is obtained.

Step 3—Add approximately 1 g of drug substance to the purified water solution and mix for 5 additional minutes to dissolve the drug to manufacture a 1% ointment.

Step 4—Slowly incorporate the aqueous solution to the oleaginous base and mix until it congeals.

Step 5—Fill the ointment into a suitable package.

### Example 10—Sterile Solution for Subcutaneous or Intramuscular Administration

The drug may be incorporated into a solution for delivery via intramuscular (IM), subcutaneous (SC), or intravenous (IV) administration. Formulations designed to deliver active drug substances via the IM or SC route will generally have similar concentrations and volumes of administration. Formulations intended to provide 1 L of drug formulation for SC or IM administration are shown here:

Formulation 19—10% (100 mg/mL)·
Drug Substance—100 g (active)
Phosphate Buffer solution—1 L (solvent)
pH adjustment—0.1 N NaOH or 0.1 N HCl (pH adjustment)
   Formulation 20—5% (50 mg/mL)
Drug Substance—50 g (active)
Phosphate Buffer solution—1 L (solvent)
pH adjustment—0.1 N NaOH or 0.1 N HCl (pH adjustment)
   Formulation 21—1% (10 mg/mL)·
Drug Substance—10 g (active)
Phosphate Buffer solution-1 L (solvent)
pH adjustment—0.1 N NaOH or 0.1 N HCl (pH adjustment)
   Formulation 22—0.1% (1 mg/mL)
Drug Substance˜1 g (active)
Phosphate Buffer solution—1 L (solvent)
pH adjustment—0.1 N NaOH or 0.1 N HCl (pH adjustment)
   Formulation 23-0.05% (500 μg/mL)
Drug Substance—0.5 g (active)
Phosphate Buffer solution—1 L (solvent)
pH adjustment—0.1 N NaOH or 0.1 N HCl (pH adjustment)

Step 1—Slowly add the drug substance to the buffer solution while stirring.

Step 2—Continue stirring until a clear solution is obtained.

Step 3—Measure the pH of the solution, and adjust to a pH of 6.5-7.5 using the dilute HCl or NaOH solution.

Step 4—Sterile filtration using a 0.22 μm filter, and fill into a sterile syringe for a pre-filled syringe drug-device combination.

The above disclosure contains various examples of peptoid compound compositions and methods of use thereof. Aspects of these various examples may all be combined with one another, even if not expressly combined in the present disclosure, unless they are clearly mutually exclusive.

In addition, various example materials are discussed herein and are identified as examples, as suitable materials, and as materials included within a more generally described type of material, for example by use of the term “including” or “such-as.” All such terms are used without limitation, such that other materials falling within the same general type exemplified but not expressly identified may be used in the present disclosure as well.

Furthermore, unless it is otherwise clear that a single entity is intended, terms such as “a,” “an,” and “the” are not intended to refer to only a singular entity and include the general class of which a specific example is described for illustration. In addition, unless it is clear that a precise value is intended, numbers recited herein should be interpreted to include variations above and below that number that may achieve substantially the same results as that number, or variations that are “about” the same number. Finally, a derivative as disclosed herein may include a chemically modified molecule that has an addition, removal, or substitution of a chemical moiety of the parent molecule.

It is understood the use of the alternative (e.g., “or”) herein is taken to mean either one or both or any combination thereof of the alternatives. The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

Various compositions may be identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The invention claimed is:

1. A composition for use in preventing or treating an infection in a wound in skin of a subject, the composition comprising one or more peptoid compounds in an amount of effective to prevent or treat the infection in the wound.

2. The composition of claim 1, wherein the one or more peptoids are selected from:
a peptoid compound H-Ntridec-NLys-Nspe-Nspe-NLys-NH₂;
a peptoid compound H-Ntetradec-NLys-Nspe-Nspe-NLys-NH₂; and
a combination thereof
wherein each peptoid is in an amount of effective to prevent or treat the infection in the wound.

3. The composition of claim 1, wherein the wound is an acute wound, a chronic wound, a cut, an abrasion, a surgical incision, a skin graft, a traumatic wound, a hypertrophic scar, a diabetic ulcer, a venous ulcer, a pressure sore, a surgical wound, a post-operative wound, a burn wound, skin or tissue damage caused by radiation treatments, or any combination thereof.

4. The composition of claim 1, wherein the composition is effective to decrease wound healing time, decrease wound closure time, promote skin graft integration, reduce scar tissue formation, reduce scar visibility, reduce or prevent wound infection, reduce or prevent wound bleeding, reduce appearance of hypertrophic scars, reduce formation of contractures in burn treatment, improve outcomes for cosmetic surgery or reconstructive surgery, improve tendon or ligament repair after orthopedic surgery, improve wound healing in patients with weakened immune systems, improve in the healing of skin and tissue damage caused by radiation treatments, reduce scar formation, or any combination thereof, following administration of the composition to a wound in skin of a subject.

5. The composition of claim 1, wherein the composition is effective to decrease wound closure time by up to 10%, 20%, 30%, 40%, 50%, 60%, 70%, 80%, 90%, 95%, 99%, or more.

6. The composition of claim 1, wherein the composition is effective to decrease wound healing time by up to 10%, 20%, 30%, 40%, 50%, 60%, 70%, 80%, 90%, 95%, 99%, or more.

7. The composition of claim 1, wherein the infection is an infection of an unidentified pathogen.

8. The composition of claim 1, wherein the infection is a bacterial infection, a fungal infection, a viral infection, or any combinations thereof.

9. The composition of claim 8, wherein the bacterial infection is an infection of a pathogen selected from Staphylococcus aureus, methicillin-resistant Staphylococcus aureus (MRSA) methicillin-susceptible Staphylococcus aureus (MSSA), coagulase-negative staphylococci, Klebsiella pneumoniae, Pseudomonas aeruginosa, Stenotrophomonas maltophilia, Citrobacter freundii, Streptococcus pneumoniae, Streptococcus pyogenes, Acinetobacter baumannii, Enterococcus faecium, Enterobacter cloacae, and any combinations thereof.

10. The composition of claim 8, wherein the fungal infection is an infection of a pathogen selected from Candida krusei, Candida albicans, Paecilomyces variotti, Mucor racemosus, Rhizopus arrhizus, Rhizopus oryzae, Aspergillus fumigatus, Aspergillus niger, and any combinations thereof.

11. The composition of claim 8, wherein the viral infection is an infection of a pathogen selected from Coronaviruses (e.g., SARS-1, SARS-COV-2, Human beta Coronavirus), MERS, Influenza virus (e.g., Influenza H1N1, H3N2, Influenza H1N1, H3N2), Herpes Simplex Virus 1, Herpes Simplex Virus 2, Hepatitis B, Hepatitis C, Chikungunya (CHIKV), Dengue Fever virus, Ebola (EVD), HSV-1 acyclovir-resistant strains, Respiratory Syncytial Virus (RSV), Rift Valley Fever Virus (RVFV), Zika (ZIKV), and any combinations thereof.

12. The composition of claim 1, wherein the composition is effective to prevent, decrease, or inhibit decrease a biofilm in the wound.

13. The composition of claim 1, formulated for topical administration, transdermal administration, transmucosal administration, intraperitoneal administration, subcutaneous administration, intramuscular administration, or intravenous administration to the subject.

14. The composition of claim 1, formulated as a cream, an ointment, a lotion, a gel, a paste, a liniment, a spray, a patch, a transdermal patch, a foam, a serum, a powder, a mousse, a balm, liposomes, a hydrogel, a microemulsion, a nanoemulsion, a salve, a salve stick, or any combinations thereof.

15. The composition of claim 1, wherein the effective amount is from 1-1000 mg/day, 25-750 mg/day, 50-500 mg/day, or 100-400 mg/day.

16. The composition of claim 1, formulated for administration one, two, three, or four times per day, once per week, once every two weeks, or once per month.

17. A method of preventing or treating an infection in a wound in skin of a subject, the method comprising:
administering the composition of claim 1, in an amount effective to prevent or treat the infection in the wound.
